SEQUENCE LISTING (72 sequences)

SEQ ID NO: 1 - DNA, 16531 bp, Artificial sequence
  Plasmid pSMAI290.
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaagatctgttttagagctagaaatagcaagttaaaataaggctagtccgttatcaacttgaaaaagtggcaccgagtcggtgctttttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaataagcttcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatggacaagaagtatagcatcgggctggacattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtacaaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgaccatatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgacccccccaagaagaagcgcaaggtctgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 2885 bp, Trichoderma reesei
ttagtccgacgccttcgagtccagccgcgtgagaatatccaaaagcacggaaaagtcgctcctggctcgcgtagccaagggtgaatcctggttgcgatggatgatggccagcaaactagctccgataaggcgaatcttttcaacctgcagcatccagacaacataatcaggacagattagcaacaagtgccgaggcttgatgatatacatatccgcagggatataaagagagagggggggtcgtcgaagaaaagggtgtgcagaaaagagaagagacaaggaaacgactcacgtttggctcgccattggcctgcaggccccaaaagggcgcctcgttcatcaccctcagcatgtctcgcacaatctccgactttctgagatcagcctgctcaacgccaggagcaaaataagagaggtcttcgaatttctgcgtaattaccatgcgcagacagtgaaacgtcacctgcaggttgatgcactgtatcacgtactgcttggactcggcagacccgttgccttctgccatcgctgccatcgccagagtgcacgactggaggtacggcgggagatcgtccaagcaggtgatgaagcggacatagagagaatcgagatgctgcctgtcggcggagggcagcacatggttcgggggcatggtccctcgaaagtgctgcatcatctgatcttgcagcacgcggatttccagcagcaaatcagccgcggactgccagagccgcacatttgcgttgaagccggatatgaagctcttccggggcggctcggacgccgtgctcaggaactcgtcctcgatacccgacgggtatagcgtggtgatgccggcgtcgaagcagtacttgtggatggtgatgggccgattgttgagtatagcggctgacttgtcgcccaagtacaggatccaaaagcagcgacggcggaactctgcctcgatggggacgagcccctcgagggcagcctcctcgtgcagctgcatgacctgcgccaggcggatggcctcgccaaatatgtgccacgagtacttgggcttccccgcagcgtggaggcagttggagtggaagtagcgaatggcaatcgagttggccgtcggattctccaggtcggcctcgaggtgctggtgcaggcagtcccgagaggcttcgagcaagatctcagagacggattctccttcggggaaaatgtccttgggtagcatgaatgctgcctctgcgcagacggccgtgaccagggtgaaggtcgagtcggcccagggagccagcctgctgccgacggttcgcgagccgctgggctggccaaagccggcccacgactcggcagcgttgaggggggtggtgccggtggtcgggtctggcgtgagctgtgacagcggcgatggttggttgacgccaggcaagggctgggagaagatgtatgcgagcacgtcccggagggccggctcgtacaccaggggggtgagggggtagaggtagtcgaagaagaggtcgatacatcgcttcgagacgcgctctagcgtcgtgttgcgcagcgtcatggcgttggccaggtgtatccatcgctggcgcgtcgagatgttctggatggtggagaggccgggcaggtcgcccagcggctgccggggcaggccgttgtcaatgctgagcggctcgacgggctcccaggccgcgtcgggcgacagcgacgacgaggcgaagggctgcagcgcggctactgcggggccggagaaggtcagcggcggcggcatctggccgggtcgagccgcggtggagagcgagctcggatcaggaggcggctggccgggctggtcgctcttcttcctcgctttggggccgcgtcgcgccaggggcaggtcgaaagtgcactgcacgtgggcatcgcggcagtgcgtgcagctatcgacagcgctgaggtactcgcactttgggcggggaagagcattggtcagtggtggcaaggagagagtatccaggtgtaagacgggggagtagcgcagcatggaagacgaggtcggtgttggggtgaggctgcagcagtatgaacgagttgtgaaggcagtgcaatacaatacacacaactctcaacacaacacaacgcttgccatgtatgctgatgtattgtatgtatgctttgtacgcatgacagacacgcaggagaagaggacgggaggagggggaacgggaacggggagaggagacacgggcagtaaggacatatgaagagcagggaccagggaccaggagacggggaggaggaggaggaggaggagcagcagcatcagcagcagcaggaggaggagcaatgacaacagagatgcaagcaacagcaaaggggctagactgaccttggccttgcgccggcggcagcggtcgcaggctctgctcaccagacggcctctgggatggtggtggaacgaggttgcagccgacacgagtgagttctgcggcggcgtggttgaggcggttggcgagccgggctgcgaggccgaggcagactcgggcgaggtggtgagggcgtggacagcgccagcgccagcgccagcagctggagggccgcctgcagctgcagctacagagccctgggccggagctgctgagcccatcgccacggagcctgtccgggtgcctggaagccctggaaggcctggagggcctgtagagctggagcctgcagcgcctgtgtggccgaggagccacggggggcagcgttagttgggggggccgcactgcactgattgcgctgcactgcgttccagtgccacccacgcgggctggcagctgggagggaacgcgagcggacgggacgggactcaacgcacctgtgtctgcacccgcagcaaccgccgcgccgccagctgctgccgcggccgctgtggccat

SEQ ID NO: 3 - DNA, 70 bp, Artificial sequence
  Primer oNJ336
ccgtgttcgattcacggatgatgcatggccaggtgtatccatcgcgttttagagctagaaatagcaagtt

SEQ ID NO: 4 - DNA, 70 bp, Artificial sequence
  Primer oNJ338
ccgtgttcgattcacggatgatgcagccgacacgagtgagttctggttttagagctagaaatagcaagtt

SEQ ID NO: 5 - DNA, 70 bp, Artificial sequence
  Primer oNJ340
ccgtgttcgattcacggatgatgcaccccggaagagcttcatatcgttttagagctagaaatagcaagtt

SEQ ID NO: 6 - DNA, 21 bp, Artificial sequence
  Primer oNJ260
gattgtgtggtgtctgccagc

SEQ ID NO: 7 - DNA, 16545 bp, Artificial sequence
  Plasmid pNJOC502
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcatggccaggtgtatccatcgcgttttagagctagaaatagcaagttaaaataaggctagtccgttatcaacttgaaaaagtggcaccgagtcggtgctttttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaataagcttcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatggacaagaagtatagcatcgggctggacattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtacaaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgaccatatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgacccccccaagaagaagcgcaaggtctgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 16545 bp, Artificial sequence
  Plasmid pNJOC503
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcagccgacacgagtgagttctggttttagagctagaaatagcaagttaaaataaggctagtccgttatcaacttgaaaaagtggcaccgagtcggtgctttttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaataagcttcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatggacaagaagtatagcatcgggctggacattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtacaaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgaccatatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgacccccccaagaagaagcgcaaggtctgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 16545 bp, Artificial sequence
  Plasmid pNJOC504
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaccccggaagagcttcatatcgttttagagctagaaatagcaagttaaaataaggctagtccgttatcaacttgaaaaagtggcaccgagtcggtgctttttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaataagcttcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatggacaagaagtatagcatcgggctggacattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtacaaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgaccatatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgacccccccaagaagaagcgcaaggtctgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 19 bp, Artificial sequence
  Primer oNJ456
gcctgcgtttctccgatcc

SEQ ID NO: 11 - DNA, 19 bp, Artificial sequence
  Primer oNJ459
caacctgcagcatccagac

SEQ ID NO: 12 - DNA, 20 bp, Artificial sequence
  Protospacer PS4
ggatccaaaagcagcgacgg

SEQ ID NO: 13 - DNA, 20 bp, Artificial sequence
  Protospacer PS6
tggccaggtgtatccatcgc

SEQ ID NO: 14 - DNA, 20 bp, Artificial sequence
  Protospacer PS9.1
ccccggaagagcttcatatc

SEQ ID NO: 15 - DNA, 86 bp, Artificial sequence
  Primer oNJ344
tgcccggcctctccaccatccagaacatctcgacgcgccaaagcttacgccatgacgctgcgcaacacgacgctagagcgcgtctc

SEQ ID NO: 16 - DNA, 86 bp, Artificial sequence
  Primer oNJ346
ctcgcagcccggctcgccaaccgcctcaaccacgccgccgaagctttgcaacctcgttccaccaccatcccagaggccgtctggtg

SEQ ID NO: 17 - DNA, 86 bp, Artificial sequence
  Primer oNJ348
gggtatcgaggacgagttcctgagcacggcgtccgagccgaagcttcggcttcaacgcaaatgtgcggctctggcagtccgcggct

SEQ ID NO: 18 - DNA, 85 bp, Artificial sequence
  Primer oNJ499
ggtggtggaacgaggttgcagccgacacgagtgagttctgcgtcagcgtggttgaggcggttggcgagccgggctgcgaggccga

SEQ ID NO: 19 - DNA, 90 bp, Artificial sequence
  Primer oNJ500
ggtggtggaacgaggttgcagccgacacgagtgagttctgcgtcggcgtagttgaggcggttggcgagccgggctgcgaggccgaggcag

SEQ ID NO: 20 - DNA, 100 bp, Artificial sequence
  Primer oNJ501
ggtggtggaacgaggttgcagccgacacgagtgagttctgcgtcggcgtggttgaggcgattggcgagccgggctgcgaggccgaggcagactcgggcga

SEQ ID NO: 21 - DNA, 110 bp, Artificial sequence
  Primer oNJ502
ggtggtggaacgaggttgcagccgacacgagtgagttctgcgtcggcgtggttgaggcggttggcgagcagggctgcgaggccgaggcagactcgggcgaggtggtgagg

SEQ ID NO: 22 - DNA, 120 bp, Artificial sequence
  Primer oNJ503
ggtggtggaacgaggttgcagccgacacgagtgagttctgcgtcggcgtggttgaggcggttggcgagccgggctgcgaagccgaggcagactcgggcgaggtggtgagggcgtggacag

SEQ ID NO: 23 - DNA, 120 bp, Artificial sequence
  Primer oNJ504
ggtggtggaacgaggttgcagccgacacgagtgagttctgcgtcggcgtggttgacgcggttggcgaaccgggctgcgaagccgaggcagactcgggcgaggtggtgagggcgtggacag

SEQ ID NO: 24 - DNA, 120 bp, Artificial sequence
  Primer oNJ505
ggtggtggaacgaggttgcagccgacacgagtgagttctgcgtcgtcgtagtttaggaggtcggcaagctgggatgtgaagccgaggcagactcgggcgaggtggtgagggcgtggacag

SEQ ID NO: 25 - DNA, 24 bp, Artificial sequence
  Primer oNJ474
cagtaaggacatatgaagagcagg

SEQ ID NO: 26 - DNA, 100 bp, Artificial sequence
  Primer oNJ567
cgaggttgcagccgacacgagtgagttctgcgtcggcgtggttgaggcggttggcgagccgggctgcgaagccgaggcagactcgggcgaggtggtgagg

SEQ ID NO: 27 - DNA, 80 bp, Artificial sequence
  Primer oNJ568
gccgacacgagtgagttctgcgtcggcgtggttgaggcggttggcgagccgggctgcgaagccgaggcagactcgggcga

SEQ ID NO: 28 - DNA, 130 bp, Artificial sequence
  Primer oNJ569
ggtggtggaacgaggttgcagccgacacgagtgagttctgcgtcggcgtggttgaggcggttggcgagccgggctgcgaggccgaggcacactcgggcgaggtggtgagggcgtggacagcgccagcgcc

SEQ ID NO: 29 - DNA, 140 bp, Artificial sequence
  Primer oNJ570
ggtggtggaacgaggttgcagccgacacgagtgagttctgcgtcggcgtggttgaggcggttggcgagccgggctgcgaggccgaggcagactcgggcgtggtggtgagggcgtggacagcgccagcgccagcgccagca

SEQ ID NO: 30 - DNA, 110 bp, Artificial sequence
  Primer oNJ571
cgaggttgcagccgacacgagtgagttctgcgtcggcgtggttgaggcggttggcgagccgggctgcgaggccgaggcacactcgggcgaggtggtgagggcgtggacag

SEQ ID NO: 31 - DNA, 120 bp, Artificial sequence
  Primer oNJ572
cgaggttgcagccgacacgagtgagttctgcgtcggcgtggttgaggcggttggcgagccgggctgcgaggccgaggcagactcgggcgtggtggtgagggcgtggacagcgccagcgcc

SEQ ID NO: 32 - DNA, 120 bp, Artificial sequence
  Primer oNJ573
cagcggtcgcaggctctgctcaccagacggcctctgggattgtggtggaacgaggttgcagccgacacgagtgagttctgcgtcggcgtggttgaggcggttggcgagccgggctgcgag

SEQ ID NO: 33 - DNA, 16195 bp, artificial sequence
  Plasmid pAT3630
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcgctacataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtacccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaagagatttcgactagttatgtacctggagtttggatataggaatgtgttgtggtaacgaaatgtaagggggaggaaagaaaaagtcgtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtaccaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgacgactttttctttcctcccccttacatttcgttaccacaacacattcctatatccaaactccaggtacataactagtcgaaatctctttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccgggaagatctcatggtcatagctgtttccgttaattaatggttcacttctctttagaaatcaactgtgggttttgctttttgcttcattctctttgtcttctccatctttgatcaaatcctggactttctcaatccccagctaattcaatcatagtcagttttctatttttattatttctttttcttttgaaatgtgattaacaaccagtccgttatatatcttgtacccagattacgcccaactcgtgctcctcagccacaaagatactcaattgatagccaagatacatacataccacaaagtaaggactccatgcattgagtattactcatcgtattctagactactccaaaactcagcacatagacaaacaatacgaacctcgtctaggggtgattcagaggcggcaaagcggggttttcgcatttgatgttcctggcacttatgtaagcccacgcttcccgctcaactaaaccatcagccaatcagactgctcagatttatcttttgaagggtaaataaatcattgtaaagaagaacaagtggcttgcttgtcaagcaatggcatcattggtctagtggtagaattcgtcgttgccatcgacgaggcccgtgttcgattcacggatgatgcaggaatttctactcttgtagatggcgcgcctttttttttgagcatttatcagcttgatatagaggtaggaatgtatggaggtgcagaatggctattttgttattggagcgggttcgaaacggagggcaggagactttttctaaatacgtcacgtgatatagagctgctttaattaacgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatccaatgcgcgattcaagacattaaaaatggaaaatttcaaaaaattgcgcccgcagcgcgtgcatacaaaattcatcccaatactcctcgtgtactgtgtaagcgcccactaggtaatatgacatgattacgaattcgagctcggtacccggccggggatcctctagacgtggagttaccagtgattgaccaatgttttatcttctacagttctgcctgtctaccccattctagctgtacctgactacagagtagtttaattgtggttgaccccacagtcggaggcggaggaatacagcaccgatgtggcctgtctccatccagattggcacgcaatttttacacgcggaaaagatcgagatagagtacgactttaaatttagtccccggcggcttctattttagaatatttgagatttgattctcaagcaattgatttggttgggtcaccctcaattggataatatacctcattgctcggctacttcaactcatcaatcaccgtcataccccgcatataaccctccattcccacgatgtcgtccaagtcgcaattgacttacggtgctcgagccagcaagcaccccaatcctctggcaaagagactttttgagattgccgaagcaaagaagacaaacgttaccgtctctgctgatgtgacgacaacccgagaactcctggacctcgctgaccgtacggaagctgttggatccaatacatatgccgtctagcaatggactaatcaacttttgatgatacaggtctcggtccctacatcgccgtcatcaagacacacatcgacatcctcaccgatttcagcgtcgacactatcaatggcctgaatgtgctggctcaaaagtacaactttttgatcttcgaggaccgcaaattcatcgacatcggcaataccgtccagaagcaataccacggcggtgctctgaggatctccgaatgggcccacattatcaactgcagcgttctccctggcgagggcatcgtcgaggctctggcccagaccgcatctgcgcaagacttcccctatggtcctgagagaggactgttggtcctggcagagatgacccccaaaggatcgctggctacgggcgagtataccaaggcatcggttgactacgctcgcaaatacaagaacttcgttatgggtttcgtgtcgacgcgggccctgacggaagtgcagtcggatgtgtcttcagcctcggaggatgaagatttcgtggtcttcacgacgggtgtgaacctctcttccaaaggagataagcttggacagcaataccagactcctgcatcggctattggacgcggtgccgactttatcatcgccggtcgaggcatctacgctgctcccgacccggttgaagctgcacagcggtaccagaaagaaggctgggaagcttatatggccagagtatgcggcaagtcatgatttcctcttggagcaaaagtgtagtgccagtacgagtgttgtggaggaaggctgcatacattgtgcctgtcattaaacgatgagctcgtccgtattggcccctgtaatgccatgttttccgcccccaatcgtcaaggttttccctttgttagattcctaccagtcatctagcaaggcggccgcagctagcacaattgaggcatccccactaccgcattaagacctcagcgcggccgcaaatttaaataaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 34 - DNA, 21 bp, artificial sequence
  twenty-one base-pairs protospacer
acgatggtgctgatggctact

SEQ ID NO: 35 - DNA, 6783 bp, Aspergillus
  misc_feature (1)..(6783): The wA locus
atagtctccactgtcgcatgtggtagtaactctatcttggtataaatcatacttaatatctagccaatcgctttgggaaaacaatttgatcagcatctctgcgactcttgactggcactttcttgacccaacatggaggggccacgcggcgtctatctcttcggagaccagacaagtgatttcgacgccggcttacgtcgcctcctacaagtaaagaataacacaattgttgcatcgttcttccagagatgctttcacgctttgcgccaagagatcgcgaggctttcaccatctgaacggaagatcttcccccggtttacgagcatagtggatctactggcgcgtcaccgggagtcagaccctaatccggctctggagagtgcgttgacctgtatctatcaattgggatgctttataaagtacgtgtaactgcagatcctgacccgtttgaacgagcctaacctgagatagctactacggagaccttggaaacgtgtacccatctgcttcagactgccatatagttggcctgtgcgcgggtcttcttagttctgcagctgtaagctgttcgaacaatgttggagaattgctccccgctgcggttgaagcggtggtggtagctctccgacttggtctatgcgtccttaaagttcgagagctggtgagctctgaccaagcgtcgtcaacaagctggtcagtcttgatttcagggattagcgagaaagatgcctcgcagcttataggagaattcactgctgaacgggtaagtcaattgatctgaaatagtttgcaggacagaatgttctaaccactggataaaggcaattcctccttcatccaaaccgtatatcagtgcggtgggatataacagtataaccatcagcgcaccgcctaaggtccttgatgatttaattgattctaggctgtctaagagccataagccggtgagggcgcaaatccatggtccttaccatgcagcacatctgtactatggccgagatgtcgacaggatcatcgaaagctgccataatgaggtcgtttcaaactacacaccccgtatccccgtactatcaagtactacgggacagccgatagaggccaaacacatgaaagatctacttaaggccgcccttgaagagattctactacgtcaactatgctgggagaaagtgaccgatgcctgctattccatattaaaaactgctcgtcatcaaccatgcaagttgttcccaatttcaagcactgcgacacaaagcttgtttacagctcttacgaaagccgggataaccgacatcgaagtggaaaatgggctcggagatgttcccacgaacccgaaggacaaccttaacatcagcggcagggcggactgctccaagatagctatcattggcatgtctggacgattcccagaagctgatggcacagagagtttctgggaccttctgtataatggcctcgatgtacaccggaaggtgcctgcagagcgttgggatgttgatgcccacgttgatcctaccggaacaaaacggaacaccagcaaggttccatacggatgctggataaacgaaccggggttatttgacccccgcttcttcaatatgtcgccacgcgaagccctccaggcagatcccgctcaaagacttgcattgctcacggcctatgaagctcttgaaatggccggctttatccccgacagcaccccttctacacagagggatcgagtcggcctcttctatggaatgactagcgatgactatcgggagataaatagtggtcaagatattgatacttactttatccctggtgggaatcgtgctttcacacctggccggataaactactatttcaagttcagtgggcccagcgtcagcgttgatacagcttgttcttcaagtcttgcggctattcatatggcttgcaattcgatctggagaaatgattgcgatgctgctattgctggaggtgtcaatatattgacaaaccctgataaccatgccggtcttgaccgtggccatttcctgtccagaaccgggaattgcaacacatttgacgatggtgctgatggctactgtagagcagatggagtgggtacaatcattctcaagcggctggaagacgctcaggcggacaacgatccaatcctcggtgtgatcaatggagcctataccaatcattcggcagaagcagtctcgattacccgccctcatgttggcgcacaagcgtttatctttaataagctattgaacgatgccaatatcgaccctaaggacgtcagctacgttgaaatgcatggaactggtactcaagctggggatgcggtggaaatgcaatcggtcttggatacgtttgctcccgactaccgccgtggaccaggacagtctctccatcttggttccgccaaagcaaatgttgggcatggagagtcagcatctggtgtaactgcacttgtgaaagtgctgctaatgatgaagaagaataccataccccctcattgtggtataaagactaagatcaaccacaacttccccacggatctcgcgcaacgaaatgtccacattgcctttcaacctaccccttggaacagaccggcttccggaaagcggcagtgcttcattaacaacttttcggcggctggtggaaataccgctcttttgatggaagacgctccaatcgctgaggttaaggggcaggacactcgacctgttcacgttgtgtctgtatcggcacgatcccagagtgcgctcaaaaacaacatcaactctctcgtaaaatacatcgacgaacaaggaaggtcattcaatgtgaacgaggcagactttatcccaagcttggcatacaccaccacagcacggcgtatccatcacccattccgtgtcacagctatcgggtctagtttgcaggagctgcgtgactcacttaacaacagctctcgtctggaaagctttacccctgtccctgcgacggcccctggcgtagggttcgtgttcgctggccaaggagctcagcacaccggaatgggaaggcaactatacgaaaaatgctctcaattccgggcaacaatgcagcacttcgattgcattagtcaaaaccaagggtttccttcgatccttcccttggttgacggaagcgtgcccgtggaggagctgggccctatcgtgacacagctcggcaccacatgtcttcagatggctttggtcaactattggggttcactaggtataaaacctgcgttcgttcttgggcatagtctcggggagtttgctgctttgaataccgcaggagtattatcgacttccgataccatctacctttgtggccgtcgggctaccctccttacagaatactgccaggttgggacacacgccatgctggctgtcaaggcttcctacccccaggtcaagcagttactgaaagaaggtgtggatgaagttgcctgtgtcaactcacccagtgagacagtcgtcagtggcctcaccgctgatattgatgacttggctcaaaggtgttccactgaaggttggaagtccactaaactaagggtaccgttcgctttccattctgcccaagttactccaattcttgaacggtttcaagaagaggcccagggtgtcacgttccgtaagccgtcgttaccgtttgtttcctcactccttggggaagtcatcaccgaatctaattacgatgtcctgggagctcaatatatggtgaagcagtgccggaagtcggtgaacttccttggtgctcttgaggccaccagatatgcgaaattgatgactgataagactgtctggctggaagttggtgcccataccatttgctctggtatgatcaaagcaacattcggtccccaggttaccactgtggcatctcttcgccgagaggagaatgcatggaaggtcctctccaatagtctatcggcccttcatttggctggcattgatattaattggaaagaatatcatcaagacttcagctccagccaccaggtgctcccacttccttcttacaagtgggatctcaagaactactggataccctacactaacaatttctgccttacgaagggtgctccccaaactgcaattcaagctgcaccacaaactacattcctgaccactgctgcgcaaaaggttgttgagagtcgcgacgacggtacaacagcgactgtcgtggtgcaaaatgacatcgctgatcctgagttgaaccgtgttatccaaggtcacaaggtcaatggagccgcactttgcccatcggtaagtattgcatgcattgccagactatcttgtgttataattcggctacttacgtattgcctagtcactctacgcagatattgcccagacacttggagagtatcttattgagaaatacaaacccgagttcaaagatcttggtctcgatgtgtgtgacatggtcgtaccgaagccactcatcgcgaagggaggagagcagctctttagagtctctgctattgctaattgggctgagaagaaggcttcagttcaagtatacgccgttaatgctgacggcaaaaagaccgtggatcatgcgtattgtacggtgaagttctttgataccaatgcctccgagctcgagtggaagagaatctcgtacctggtcaagagaagcatcgacagtcttcaccagaatgcggagacaggggaggctcaccgtatccagcgaggaatggtctataaacttttcagcgcgttggtcgattatgatgaaaatttcaagtcgattcgcgaggttatcctggacagcgacaataatgaggccaccgctcgtgtcaaattccaagcaccgccaggaaatttccaccgaaacccattctggattgacagtttcggtcacttgtccggattcattatgaatgcgagcgacgcgaccgactctaagaaccaagtatttgttaaccatggatgggattcgatgcgttgcctgaagaagttctcgcctgatgtcacttatcgcacttatgtgaggatgcagccatggcaaaacaacatttgggctggagatgtttatatctttgagggcgacgatattattgctgtcttcggaggtgtgaaggtgggtacctcactactgattttggttcctgcttactgacatgataattagttccaagcactggcacgcaagatacttgacactgttcttccccctgttggcggttcaaaggcaccaattacagcgaaatcaccacctccagctcgcactcagaaggccaacaccggcgccaagacccgtcctaaagcacctgttccttccaagtcgttcaccaaatcttctgggccgagtgttgtcgtacgcgcactcagcattctggcctcagaagttggcctggcagagtctgaaatctcagacgacatggtgtttgcggactacggtgtagactcactcctctcccttacagttactggcaggtatcgtgaagagttgaacctcgatttggactcctctgtgtttaccgatcatccaactgtcaacgacttcaagcggctcatcgcccaagtgagtccttcagagagccatgatggttcctccagtgaacaagagtcgaatttctctttcaacggtggcgagtcctcaagcgcaagcacacctgacataacgtcaccgccgaatgagaaggtagctcaagtcgagcaaaacggcaccatgaaggaaatccgtaacatcatggcggaggagatcggtgtacccgcagaagagatcgaccctgacgagaacttgggagagatgggtatggactcgcttctctcccttactgttcttggaagaatacgggagactttggacatggacctgccaggagagttcttcatcgaaaaccagaccctcaatgatatagaggtggctttggacctaaaacccaagactacctctgctccaattcctatgccagagccagtgaaattccctgaagctatccacgacctccagccaaagcttgctcaacatcccaaggccacatccatcctgttacaaggaaaccccaggacagcaacaaagacgttattcttgtttcctgacggctctggctcagctacatcttacgctaccatccccggactctctcctgacgtctgcgtttacgggttgaattgcccatatatgaagacacctgagaagctcaaatgcagcctagatgaactcactgcgccctatgtagcagagattcgtcgtcggcaacccaagggtccttacagcttcggtggctggtcagcaggagggatctgtgcatatgatgcggcacgccatctaatgtttgaggaaggtgaacaagtcgaccgcttgcttctccttgatacccccttccccatcggcctcgagaagctgccgcagagattgtacggcttcttcaactctatcggtctcttcggtgaaggtaaaacggcaccaccctcctggctcctaccccacttcctagcctttatcgacgctctcgacgcatacaaggccgcgccccttccattcaaagacgagaaatgggccaagaaactgcccaagacttatatcatctgggccaaggacggtgtttgcggtaagccgggagatccccggcctgatcccccgacagacggttccaaggatcccaaggagatggtctggcttcttaatgaccggaccgatctgggacctaacaagtgggatacattggttggacctgagaatattggtggaatcacagtaatggaagatgctaatcattttacgatgacgaagggcgaaaaagcgaaagagttgtctacatttatggctaacgccatggcttaa

SEQ ID NO: 36 - DNA, 66 bp, artificial sequence
  The protospacer with its extension sequences used for cloning (oAT3858)
aatttctactcttgtagatacgatggtgctgatggctacttttttttttgagcatttatcagcttg

SEQ ID NO: 37 - DNA, 20 bp, artificial sequence
  Primer oAT4025
gcccgttcgagagcatgatc

SEQ ID NO: 38 - DNA, 16208 bp, artificial sequence
  Plasmid pAT3720
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcgctacataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtacccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaagagatttcgactagttatgtacctggagtttggatataggaatgtgttgtggtaacgaaatgtaagggggaggaaagaaaaagtcgtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtaccaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgacgactttttctttcctcccccttacatttcgttaccacaacacattcctatatccaaactccaggtacataactagtcgaaatctctttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccgggaagatctcatggtcatagctgtttccgttaattaatggttcacttctctttagaaatcaactgtgggttttgctttttgcttcattctctttgtcttctccatctttgatcaaatcctggactttctcaatccccagctaattcaatcatagtcagttttctatttttattatttctttttcttttgaaatgtgattaacaaccagtccgttatatatcttgtacccagattacgcccaactcgtgctcctcagccacaaagatactcaattgatagccaagatacatacataccacaaagtaaggactccatgcattgagtattactcatcgtattctagactactccaaaactcagcacatagacaaacaatacgaacctcgtctaggggtgattcagaggcggcaaagcggggttttcgcatttgatgttcctggcacttatgtaagcccacgcttcccgctcaactaaaccatcagccaatcagactgctcagatttatcttttgaagggtaaataaatcattgtaaagaagaacaagtggcttgcttgtcaagcaatggcatcattggtctagtggtagaattcgtcgttgccatcgacgaggcccgtgttcgattcacggatgatgcaggaatttctactcttgtagatacgatggtgctgatggctacttttttttttgagcatttatcagcttgatatagaggtaggaatgtatggaggtgcagaatggctattttgttattggagcgggttcgaaacggagggcaggagactttttctaaatacgtcacgtgatatagagctgctttaattaacgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatccaatgcgcgattcaagacattaaaaatggaaaatttcaaaaaattgcgcccgcagcgcgtgcatacaaaattcatcccaatactcctcgtgtactgtgtaagcgcccactaggtaatatgacatgattacgaattcgagctcggtacccggccggggatcctctagacgtggagttaccagtgattgaccaatgttttatcttctacagttctgcctgtctaccccattctagctgtacctgactacagagtagtttaattgtggttgaccccacagtcggaggcggaggaatacagcaccgatgtggcctgtctccatccagattggcacgcaatttttacacgcggaaaagatcgagatagagtacgactttaaatttagtccccggcggcttctattttagaatatttgagatttgattctcaagcaattgatttggttgggtcaccctcaattggataatatacctcattgctcggctacttcaactcatcaatcaccgtcataccccgcatataaccctccattcccacgatgtcgtccaagtcgcaattgacttacggtgctcgagccagcaagcaccccaatcctctggcaaagagactttttgagattgccgaagcaaagaagacaaacgttaccgtctctgctgatgtgacgacaacccgagaactcctggacctcgctgaccgtacggaagctgttggatccaatacatatgccgtctagcaatggactaatcaacttttgatgatacaggtctcggtccctacatcgccgtcatcaagacacacatcgacatcctcaccgatttcagcgtcgacactatcaatggcctgaatgtgctggctcaaaagtacaactttttgatcttcgaggaccgcaaattcatcgacatcggcaataccgtccagaagcaataccacggcggtgctctgaggatctccgaatgggcccacattatcaactgcagcgttctccctggcgagggcatcgtcgaggctctggcccagaccgcatctgcgcaagacttcccctatggtcctgagagaggactgttggtcctggcagagatgacccccaaaggatcgctggctacgggcgagtataccaaggcatcggttgactacgctcgcaaatacaagaacttcgttatgggtttcgtgtcgacgcgggccctgacggaagtgcagtcggatgtgtcttcagcctcggaggatgaagatttcgtggtcttcacgacgggtgtgaacctctcttccaaaggagataagcttggacagcaataccagactcctgcatcggctattggacgcggtgccgactttatcatcgccggtcgaggcatctacgctgctcccgacccggttgaagctgcacagcggtaccagaaagaaggctgggaagcttatatggccagagtatgcggcaagtcatgatttcctcttggagcaaaagtgtagtgccagtacgagtgttgtggaggaaggctgcatacattgtgcctgtcattaaacgatgagctcgtccgtattggcccctgtaatgccatgttttccgcccccaatcgtcaaggttttccctttgttagattcctaccagtcatctagcaaggcggccgcagctagcacaattgaggcatccccactaccgcattaagacctcagcgcggccgcaaatttaaataaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 39 - DNA, 27 bp, artificial sequence
  Primer oAT4074
gtctggacgattcccagaagctgatgg

SEQ ID NO: 40 - DNA, 26 bp, artificial sequence
  Primer oAT4075
ctctgggatcgtgccgatacagacac

SEQ ID NO: 41 - DNA, 25 bp, artificial sequence
  Primer oAT4076
ccagcgtcagcgttgatacagcttg

SEQ ID NO: 42 - DNA, 120 bp, artificial sequence
  Primer oAT4070
cgtggccatttcctgtccagaaccgggaattgcaacacataagacgatggtgctgatggctactgtagagcagatggagagggtacaatcattctcaagcggctggaagacgctcaggcg

SEQ ID NO: 43 - DNA, 140 bp, artificial sequence
  Primer oAT4071
cgtggccatttcctgtccagaaccgggaattgcaacacataagacgatggtgctgatggctactgtagagcagatggagtgggtacaatcattctcaaggggctggaagacgctcaggcggacaacgatccaatcctcgg

SEQ ID NO: 44 - DNA, 160 bp, artificial sequence
  Primer oAT4072
cgtggccatttcctgtccagaaccgggaattgcaacacataagacgatggtgctgatggctactgtagagcagatggagtgggtacaatcattctcaagcggctggaagacgctcaggccgacaacgatccaatcctcggtgtgatcaatggagcctata

SEQ ID NO: 45 - DNA, 16354 bp, artificial sequence
  Plasmid pGMEr263
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaggaatttctactcttgtagatctttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaatcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 46 - DNA, 93 bp, artificial sequence
  Protospacer oligo 1228713
ttcacggatgatgcaggaatttctactcttgtagatcgttgaagccggatatgaagctttttttggctcttgggttcgaactgcccaaggccc

SEQ ID NO: 47 - DNA, 93 bp, artificial sequence
  Protospacer oligo 1228715
ttcacggatgatgcaggaatttctactcttgtagatgcgaggccatccgcctggcgctttttttggctcttgggttcgaactgcccaaggccc

SEQ ID NO: 48 - DNA, 93 bp, artificial sequence
  Protospacer oligo 1228717
ttcacggatgatgcaggaatttctactcttgtagatccccgaaggagaatccgtctctttttttggctcttgggttcgaactgcccaaggccc

SEQ ID NO: 49 - DNA, 93 bp, artificial sequence
  Protospacer oligo 1228719
ttcacggatgatgcaggaatttctactcttgtagattgcgtaattaccatgcgcagatttttttggctcttgggttcgaactgcccaaggccc

SEQ ID NO: 50 - DNA, 93 bp, artificial sequence
  Protospacer oligo 1228721
ttcacggatgatgcaggaatttctactcttgtagattatccctgcggatatgtatattttttttggctcttgggttcgaactgcccaaggccc

SEQ ID NO: 51 - DNA, 20 bp, artificial sequence
  primer 1228659
cttgcttgtcaagcaatggc

SEQ ID NO: 52 - DNA, 16374 bp, artificial sequence
  Plasmid pGMEr263proto1
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaggaatttctactcttgtagatcgttgaagccggatatgaagctttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaatcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 53 - DNA, 16374 bp, artificial sequence
  Plasmid pGMEr263proto2
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaggaatttctactcttgtagatgcgaggccatccgcctggcgctttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaatcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 54 - DNA, 16374 bp, artificial sequence
  Plasmid pGMEr263proto3
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaggaatttctactcttgtagatccccgaaggagaatccgtctctttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaatcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 55 - DNA, 16374 bp, artificial sequence
  Plasmid pGMEr263proto4
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaggaatttctactcttgtagattgcgtaattaccatgcgcagatttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaatcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 56 - DNA, 16374 bp, artificial sequence
  Plasmid pGMEr263proto5
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtaccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccggggatcctctagagtcgacctgcaggttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatgattcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattgcctgcaggcatgcaagctttctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttcggcttgcttgtcaagcaatggcatcattggtctagtggtagaattcgtcgttgccatcgacgaggcccgtgttcgattcacggatgatgcaggaatttctactcttgtagattatccctgcggatatgtatattttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaatcgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtgagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 57 - DNA, 20 bp, artificial sequence
  Primer 1228586
ttagtccgacgccttcgagt

SEQ ID NO: 58 - DNA, 20 bp, artificial sequence
  Primer 1228587
tgcgagtacctcagcgctgt

SEQ ID NO: 59 - DNA, 21 bp, artificial sequence
  Protospacer PS1
cgttgaagccggatatgaagc

SEQ ID NO: 60 - DNA, 21 bp, artificial sequence
  Protospacer PS2
gcgaggccatccgcctggcgc

SEQ ID NO: 61 - DNA, 21 bp, artificial sequence
  Protospacer PS3
ccccgaaggagaatccgtctc

SEQ ID NO: 62 - DNA, 21 bp, artificial sequence
  Protospacer PS4
tgcgtaattaccatgcgcaga

SEQ ID NO: 63 - DNA, 21 bp, artificial sequence
  Protospacer PS5
tatccctgcggatatgtatat

SEQ ID NO: 64 - DNA, 94 bp, artificial sequence
  Repair DNA 1228583
cggatttccagcagcaaatcagccgcggactgccagagccgcacaagcttcttccggggcggctcggacgccgtgctcaggaactcgtcctcga

SEQ ID NO: 65 - DNA, 94 bp, artificial sequence
  Repair DNA 1228584
tggggacgagcccctcgagggcagcctcctcgtgcagctgcatgaagcttatgtgccacgagtacttgggcttccccgcagcgtggaggcagtt

SEQ ID NO: 66 - DNA, 95 bp, artificial sequence
  Repair DNA 1228585
aggtgctggtgcaggcagtcccgagaggcttcgagcaagatctcaagctttgtccttgggtagcatgaatgctgcctctgcgcagacggccgtga

SEQ ID NO: 67 - DNA, 94 bp, artificial sequence
  Repair DNA 1228582
atcagcctgctcaacgccaggagcaaaataagagaggtcttcgaagcttcagtgaaacgtcacctgcaggttgatgcactgtatcacgtactgc

SEQ ID NO: 68 - DNA, 94 bp, artificial sequence
  Repair DNA 1228581
caacataatcaggacagattagcaacaagtgccgaggcttgatgaagcttaaagagagagggggggtcgtcgaagaaaagggtgtgcagaaaag

SEQ ID NO: 69 - DNA, 122 bp, artificial sequence
  Oligo10
tttccagcagcaaatcagccgcggactgccagagccgcacattggcgttaaagccggatatgaagctcttccggggcggctcgcacgccgtgctcaggaactcgtcctcgatacccgacggg

SEQ ID NO: 70 - DNA, 135 bp, artificial sequence
  Oligo11
tttccagcagcaaatcagccgcggactgccagagccgcacattggcgttaaagccggatatgaagctcttccggggcggctcggacgccgtgctctggaactcgtcctcgatacccgacgggtatagcgtggtga

SEQ ID NO: 71 - DNA, 150 bp, artificial sequence
  Oligo12
tttccagcagcaaatcagccgcggactgccagagccgcacattggcgttaaagccggatatgaagctcttccggggcggctcggacgccgtgctcaggaactcgtcctcgctacccgacgggtatagcgtggtgatgccggcgtcgaagc

SEQ ID NO: 72 - DNA, 111 bp, artificial sequence
  Oligo13
ctgatcttgcagcacgcggatttccagcagcaaatcagccacggactgccagagccgcacattggcgttaaagccggatatgaagctcttccggggcggctcggacgccgt

(19) United States
(12) Patent Application Publication
(10) Pub. No.: US 2022/0010305 A1
(43) Pub. Date: Jan. 13, 2022

(54) Genome Editing by Guided Endonuclease and Single-stranded Oligonucleotide

(71) Applicant: Novozymes A/S, Bagsvaerd (DK)

(72) Inventors: Nicholas Jochumsen, Lyngby (DK); Gloria Muzzi-Erichsen, Davis, CA (US)

(73) Assignee: Novozymes A/S, Bagsvaerd (DK)

(21) Appl. No.: 17/289,953

(22) PCT Filed: Oct. 30, 2019
(86) PCT No.: PCT/US2019/058913

§ 371 Date: Apr. 29, 2021

Related U.S. Application Data
(60) Provisional application No. 62/753,366, filed on Oct. 31, 2018.

(51) Int. Cl. C12N 15/11; C12N 15/10

(52) U.S. Cl. CPC C12N 15/111; C12N 15/102

(57) ABSTRACT

The present invention relates to methods for introducing one or more desired nucleotide modification(s) in a target sequence in the genome of a microorganism cell using a polynucleotide-guided endonuclease, e.g., the MAD7 enzyme isolated and described by Inscripta™ or the well-known Streptococcus pyogenes Cas9, together with a suitable guide RNA for each target sequence to be modified, to generate a site-specific cut or nick in at least one genome target sequence, followed by the repair of the cut(s) and/or nick(s) via at least one oligonucleotide capable of hybridizing with the at least one genome target sequence, thereby highly efficiently introducing the one or more desired modification(s) into the target sequence.

### REFERENCE TO SEQUENCE LISTING

This application contains a Sequence Listing in computer readable form. The computer readable form is incorporated herein by reference.

### FIELD OF THE INVENTION

The invention provides methods for modifying the genome of a host cell by employing a programmable polynucleotide-guided endonuclease enzyme, e.g., the MAD7 enzyme isolated and described by Inscripta™ or the well-known Streptococcus pyogenes Cas9, together with one or more single-stranded oligonucleotide as donor DNA.

### BACKGROUND OF THE INVENTION

The so-called CRISPR (clustered regularly interspaced short palindromic repeats) Cas9 genome editing system originally isolated from Streptococcus pyogenes has been widely used as a tool to modify the genomes of a number of microorganisms as well as higher organisms.

The programmable Cas9 enzyme has two RNA-guided DNA endonuclease domains capable of targeting specific genomic sequences. The system has been described extensively for editing genomes in a variety of eukaryotes [Doudna, J. A. and E. Charpentier, Genome editing. The new frontier of genome engineering with CRISPR-Cas9. Science, 2014. 346(6213): p.1258096], human stem cells [Paquet, D., et al., Efficient introduction of specific homozygous and heterozygous mutations using CRISPR/Cas9. Nature, 2016. 533: p. 125-129], mouse zygotes [Inui, M., et al., Rapid generation of mouse models with defined point mutations by the CRISPR/Cas9 system. Sci Rep. 2014; 4:5396], pigs [Wang, K., et al., Efficient Generation of Orthologous Point Mutations in Pigs via CRISPR-assisted ssODN-mediated Homology-directed Repair. Mol Ther Nucleic Acids. 2016 November; 5(11): e396], E. coli [Jiang, W., et al., RNA-guided editing of bacterial genomes using CRISPR-Cas systems. Nat Biotechnol, 2013. 31(3): p. 233-9], yeast [DiCarlo, J. E., et al., Genome engineering in Saccharomyces cerevisiae using CRISPR-Cas systems. Nucleic Acids Res, 2013. 41(7): p. 4336-43, [Horwitz, A. A., et al., Efficient Multiplexed Integration of Synergistic Alleles and Metabolic Pathways in Yeasts via CRISPR-Cas. Cell Syst. 2015. 1(1): p. 88-96], Lactobacillus [Oh, J. H. and J. P. van Pijkeren, CRISPR-Cas9-assisted recombineering in Lactobacillus reuteri. Nucleic Acids Res, 2014. 42(17): p. e131] and filamentous fungi such as Trichoderma reesei [Liu, R., et al., Efficient genome editing in filamentous fungus Trichoderma reesei using the CRISPR/Cas9 system. Cell Discovery, 2015. 1].

The power of the Cas9 system lies in its simplicity and ability to target and edit a specific gene of interest. In addition, it is possible to target multiple genes for modification (multiplexing) in a single reaction, to generate insertions and deletions as well as silence or activate genes. In 2012 the Cas9 protein was shown to be a dual-RNA guided endonuclease protein [Jinek, M., et al., A programmable dual-RNA-guided DNA endonuclease in adaptive bacterial immunity. Science, 2012. 337(6096): p. 816-21.].

Further development has led to the engineering of a single guide-RNA molecule that guides the endonuclease to its DNA target. The single guide-RNA retains the critical features necessary for both interaction with the Cas9 protein and targeting the desired nucleotide sequence. When complexed with the RNA molecule, the Cas9 protein binds to the target sequence and creates a double stranded break using two catalytic domains.

When engineered to contain a single amino acid mutation in either catalytic domain, the Cas9 protein functions as a nickase, a variant protein with single stranded cleavage activity. Genome editing in Clostridium cellulolyticum via CRISPR-Cas9 nickase was recently demonstrated by Xu et al. [Xu, T., et al., Efficient Genome Editing in Clostridium cellulolyticum via CRISPR-Cas9 Nickase. Appl Environ Microbiol, 2015. 81(13): p. 4423-31.].

A plethora of scientific publications and published patent applications relating to genome editing has become available. More recently, a general method for transforming a replicative plasmid carrying the S. pyogenes Cas9-encoding gene into Aspergillus niger was described by Nødvig et al. [A CRISPR-Cas9 System for Genetic Engineering of Filamentous Fungi. 2015. PLoS ONE 10(7): e0133085. doi:10.1371/journal. pone.0133085].

Many new polynucleotide-guided and programmable endonucleases have been described since the first discovery of the Cas9 enzyme, including, for example, the MAD7 enzyme isolated from Madagascar and described by Inscripta™, who released its DNA sequence and pledged on their website that the MAD7 enzyme is royalty-free for all R&D use. Even so, the MAD7 gene editing systems were recently patented by Inscripta™. MAD7 has been shown to be effective in both microbial and mammalian systems.

It has been shown that it is possible to use single-stranded oligonucleotides as donor DNA in Cas9-based genome editing. However, several studies have shown that there is a negative correlation between the distance from the cut site and incorporation of mutations using single-stranded oligonucleotides as donor DNA [Inui et al., 2014; Wang et al., 2016; Paquet et al., 2016; vide supra]. In pigs (porcine fetal fibroblasts), Wang et al. (2016) demonstrated that a mutation-to-cut distance of 11 bp resulted in a remarkable difference in homology-directed repair efficiency between two point mutations [Wang, K., et al., Efficient Generation of Orthologous Point Mutations in Pigs via CRISPR-assisted ssODN-mediated Homology-directed Repair. Mol Ther Nucleic Acids. 2016 November; 5(11): e396]. Likewise, for human induced pluripotent stem cells, Paquet et al. (2016) state that cut-to-mutation distance needs to be minimized for efficient homozygous mutation incorporation [Paquet, D., et al., Efficient introduction of specific homozygous and heterozygous mutations using CRISPR/Cas9. Nature, 2016. 533: p. 125-129]. Similarly, for mice, Inui et al. (2014) report that the distance between the modification site and gRNA target site was a significant parameter affecting the efficiency of the substitution [Inui, M., et al., Rapid generation of mouse models with defined point mutations by the CRISPR/Cas9 system. Sci Rep. 2014; 4:5396].

For directed mutagenesis or genome editing in Saccharomyces cerevisiae, Horwitz et al. (2015) report that the site targeted for cutting should as close as possible to the site of the desired mutation. Furthermore, to disrupt the Cas9 target site in the donor DNA and simultaneously improve the chances that recombination events include the desired mutation, Horwitz et al. (2015) made silent changes in the codons between the target site and the point mutation (a “heterology block”) [Horwitz, A. A., et al., Efficient Multiplexed Integration of Synergistic Alleles and Metabolic Pathways in Yeasts via CRISPR-Cas. Cell Syst. 2015. 1(1): p. 88-96].

One limitation in the usual genome editing methods is, that it may be difficult to find a proper PAM site and a good protospacer sequence in close proximity to where you intend to modify the genome and/or that incorporation of multiple silent changes in the codons between the target site and the point mutation in an open reading frame can be lead to undesired effects such as alternative splicing in eukaryotes.

### SUMMARY OF THE INVENTION

The inventors found that, contrary to what had been reported elsewhere, they were able to employ longer single-stranded oligonucleotides comprising desired nucleotide modifications as repair templates or “donor DNA” in microorganism host cells, after cutting or nicking a double stranded genomic DNA sequence in the vicinity of a target sequence to be modified using a programmable endonuclease, whereby the nucleotide modifications of the oligonucleotides would be successfully introduced into the double-stranded DNA with comparatively high efficiencies.

Accordingly, in a first aspect, the invention relates to methods for introducing one or more desired nucleotide modification(s) in at least one target sequence in the genome of a microorganism cell using a polynucleotide-guided endonuclease, said method comprising the steps of:
a) providing a microorganism host cell comprising at least one genome target sequence to be modified located in the vicinity of a protospacer adjacent motif (PAM) sequence for the polynucleotide-guided endonuclease;
b) transforming the microorganism host cell with:
   i) the polynucleotide-guided endonuclease and at least one suitable guide polynucleotide for the at least one target sequence to be modified, OR one or more polynucleotide encoding the polynucleotide-guided endonuclease and encoding at least one suitable guide polynucleotide for the at least one target sequence to be modified, and
   ii) at least one single-stranded or double-stranded oligonucleotide capable of hybridizing with the at least one genome target sequence, said oligonucleotide comprising the one or more desired nucleotide modification(s);

wherein the polynucleotide-guided endonuclease interacts with the guide polynucleotide and with the genome and cuts or nicks the genome, and wherein the at least one single-stranded or double-stranded oligonucleotide directs DNA repair across the cut or nick, thereby introducing the one or more desired modification(s) into the target sequence of the genome with an efficiency of at least:

70% when the cut or nick is located 10-20 nucleotides from the desired nucleotide modification(s),

60% when the cut or nick is located 21-30 nucleotides from the desired nucleotide modification(s),

50% when the cut or nick is located 31-43 nucleotides from the desired nucleotide modification(s),

40% when the cut or nick is located 44-52 nucleotides from the desired nucleotide modification(s), or 30% when the cut or nick is located at least 53 nucleotides from the desired nucleotide modification(s).

### BRIEF DESCRIPTION OF DRAWINGS

FIG. 1 shows a plasmid map of pSMAI290.

FIG. 2 shows a plasmid map of pNJ00502.

FIG. 3 shows a plasmid map of pNJ00503.

FIG. 4 shows a plasmid map of pNJ00504.

FIG. 5 shows an overview of the oligonucleotides used in Example 5 herein. The arrow shows the region of that gene, where the DNA is cut, including the regions that are homologous to the oligonucleotides used in this study (not to scale). Each oligonucleotide contains a mutation in the region corresponding to the PAM site to avoid re-cutting of the DNA upon recombination (indicated by a filled circle; ●). In addition to the PAM mutation, each oligonucleotide contains another mutation placed at increasing distance away from the cut site (indicated by a triangle; ▾). Furthermore, oligonucleotides oNJ504 and oNJ505 contain additional mutations (indicated by filled diamonds; ♦) to serve as “buffer mutations” to increase the likelihood of incorporation of the mutation indicated by the triangle upon recombination between the target site and the oligonucleotide. The positions of the mutations relative to the cut site are indicated in the bottom of the figure. Each oligonucleotide contains 42 unmodified nt on the 5′ side of the PAM mutation and 40 unmodified nt on the 3′ side of the mutation indicated by the triangle (▾).

FIG. 6 shows an overview of the oligonucleotides used in Example 6 herein. The arrow shows the region of that gene, where the DNA is cut, including the regions that are homologous to the oligonucleotides used in this study (not to scale). Each oligonucleotide contains a mutation in the region corresponding to the PAM site to avoid re-cutting of the DNA upon recombination (indicated by a filled circle; ●). In addition to the PAM mutation, each oligonucleotide contains another mutation placed at increasing distance away from the cut site (indicated by a triangle; ▾). The positions of the mutations relative to the cut site are indicated in the bottom of the figure. Oligonucleotides oNJ503, oNJ569 and oNJ570 contain 42 unmodified nt on the 5′ side of the PAM mutation and 40 unmodified nt on the 3′ side of the mutation indicated by the triangle. Oligonucleotides oNJ567, oNJ571 and oNJ572 contain 32 unmodified nt on the 5′ side of the PAM mutation and 30 unmodified nt on the 3′ side of the mutation indicated by the triangle. Oligonucleotide oNJ569 contains 22 unmodified nt on the 5′ side of the PAM mutation and 20 unmodified nt on the 3′ side of the mutation indicated by the triangle. Oligonucleotide oNJ573 contains 40 unmodified nt on the 5′ side of the mutation indicated by the triangle and 40 unmodified nt on the 3′ side of the PAM mutation.

FIG. 7 shows a plasmid map of pAT3630.

FIG. 8 shows a plasmid map of pAT3720, which was used to test CRISPR/Mad7 assisted delivery of mutations using single-stranded oligonucleotides in Aspergillus oryzae.

FIG. 9 shows a plasmid map of pGMEr263.

FIG. 10 shows a plasmid map of pGMEr263-proto1, which was used to test CRISPR/Mad7 assisted delivery of mutations using single-stranded oligonucleotides in Trichoderma reesei.

FIG. 11 shows a plasmid map of pGMEr263-proto2.

FIG. 12 shows a plasmid map of pGMEr263-proto3.

FIG. 13 shows a plasmid map of pGMEr263-proto4.

FIG. 14 shows a plasmid map of pGMEr263-proto5.

### DEFINITIONS

Genomic modifications: The term “genomic modification(s)” includes any modification in a genomic sequence, both non-coding or coding, at the nucleotide level. Such modifications may not alter the amino acid sequence of an encoded polypeptide or they may lead to changes in the amino acid sequence, such as, deletions, insertions or substitutions.

If an amino acid is substituted for another amino acid with similar characteristics it may be termed a conservative substitution. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Programmable polynucleotide-guided endonuclease: The term “programmable polynucleotide-guided endonuclease” or “polynucleotide-guided endonuclease” or “polynucleotide-guided nuclease” are used interchangeably herein. The term includes the so-called class-II Cas9 analogues or homologues, of which several are known and more are being discovered almost monthly as the scientific interest has surged over the last few years; a review is provided in Makarova K. S. et al, An updated evolutionary classification of CRISPR-Cas systems, 2015, Nature vol. 13: 722-736.

Cas endonuclease: The term “Cas endonuclease” means an RNA-guided DNA endonuclease associated with CRISPR that cleaves a target DNA sequence when coupled with a single guide RNA. The Cas endonuclease is guided by the single guide RNA(s) to recognize and cleave a specific target site in double stranded DNA in the genome of a cell. CRISPR-Cas systems are currently classified as Type I, Type II, and Type III CRISPR-Cas systems (Liu and Fan, 2014, Plant Mol. Biol. 85: 209-218). For purposes of the present disclosure, the CRISPR-Cas system is a Type II CRISPR-Cas system employing a Cas9 endonuclease or variant thereof (including, for example, a Cas9 nickase). The Cas9 endonuclease comprises two nuclease domains, an HNH (McrA-like) nuclease domain that cleaves the complementary DNA strand and a RuvC-like nuclease domain that cleaves the noncomplementary DNA strand. Target recognition and cleavage by the Cas9 endonuclease requires a chimeric single guide RNA consisting of a fusion of crRNA (a 20-nucleotide guide sequence and a partial direct repeat) and tracrRNA (transactivating crRNA) and a short conserved sequence motif downstream of the crRNA binding region, called a protospacer adjacent motif (PAM). In the CRISPR-Cas9 system derived from the bacterium Streptococcus pyogenes, the target DNA immediately precedes a 5′-NGG PAM. The RNA-guided Cas9 endonuclease activity creates site-specific double strand breaks, which are then repaired by either non-homologous end joining (NHEJ) or homology-directed repair (HDR). It is understood that the term “Cas endonuclease” encompasses variants thereof.

Cas-nickase: The term “Cas9 nickase” means a Cas9 endonuclease that introduces a single-strand nick into a target double stranded DNA sequence when coupled with a chimeric single guide RNA. Cas9 nickases can be generated recombinantly by inactivating one of the two nuclease domains in a parent Cas9 endonuclease (e.g., by site-directed mutagenesis). A non-limiting example of a Cas9 nickase is the Cas9 nickase in which the RuvC domain is inactivated by a D10A mutation in the Cas9 endonuclease from Streptococcus pyogenes (Sander and Joung, 2013, Nature Biotechnology 1-9). Two guide RNAs designed on opposite DNA strands are required with a Cas9 nickase to create a double stranded break.

Mad endonuclease: The term “Mad endonuclease” means an RNA-guided DNA endonuclease associated with CRISPR that cleaves a target DNA sequence when coupled with a single guide RNA. The Mad endonuclease is guided by the single guide RNA(s) to recognize and cleave a specific target site in double stranded DNA in the genome of a cell. CRISPR-Mad systems are closely related to the Type V (Cpf1-like) of Class-2 family of CAS enzymes. For purposes of the present disclosure, the CRISPR-Mad system employs an Eubacterium rectale MAD7 endonuclease or variant thereof. The MAD7-crRNA complex cleaves target DNA by identification of a protospacer adjacent motif (PAM) 5′-YTTN. After identification of the PAM, MAD7 introduces sticky-end DNA double-stranded break of 4-5 nucleotides overhang to the 3′ end of the targeted PAM which is then repaired by either non-homologous end joining (NHEJ) or homology-directed repair (HDR). It is understood that the term “Mad endonuclease” encompasses variants thereof.

Cpf1 endonuclease: The term “Cpf endonuclease” means an RNA-guided DNA endonuclease associated with CRISPR that cleaves a target DNA sequence when coupled with a single guide RNA. The Cpf endonuclease is guided by the single guide RNA(s) to recognize and cleave a specific target site in double stranded DNA in the genome of a cell. For purposes of the present disclosure, the CRISPR-Cpf system employs an Acidaminococcus sp. Cpf1 endonuclease, a Lachnospiraceae sp. Cpf1 endonuclease, or a Francisella novicide Cpf1 endonuclease or variant thereof. The Cpf1-crRNA complex cleaves target DNA by identification of a protospacer adjacent motif (PAM) 5′-TTTN for the Acidaminococcus sp. Cpf1 endonuclease and Lachnospiraceae sp. Cpf1) endonuclease, and a PAM sequence 5′-TTN for the Francisella novicide Cpf1. After identification of the PAM, Cpf1 introduces sticky-end DNA double-stranded break of 4-5 nucleotides overhang distal to the 3′ end of the targeted PAM which is then repaired by either non-homologous end joining (NHEJ) or homology-directed repair (HDR). It is understood that the term “Cpf1 endonuclease” encompasses variants thereof.

Nuclear localization signal (NLS): The programmable endonuclease encoding polynucleotide may be operably linked to one or more polynucleotides encoding nuclear localization signal(s), so the expressed endonuclease is efficiently transported from the cytoplasm to the nucleus in eukaryotic host cells. Examples of suitable nuclear localization signals include the SV40 nuclear localization signal, Aspergillus nidulans GATA transcription factor (AreA), Trichoderma reesei transcriptional regulator for cellulase and hemicellulase gene expression (XYR1), Trichoderma reesei blue light regulator 2 (blr2), Xenopus laevis oocyte Nucleoplasmin nuclear localization signal, Caenorhabditis elegans transcription factor EGL-13 nuclear localization signal, Homo sapiens transcription factor c-Myc nuclear localization signal, and Escherichia coli replication fork arresting protein (TUS-protein) nuclear localization signal.

Guide RNA: The term “guide RNA” in CRISPR-Cas9 genome editing refers to the re-programmable part that makes the system so versatile. In the natural S. pyogenes system the guide RNA is actually a complex of two RNA polynucleotides, a first crRNA containing about 20 nucleotides that determine the specificity of the Cas9 enzyme as well as the tracr RNA which hybridizes to the cr RNA to form an RNA complex that interacts with Cas9. See Jinek M. et al., A programmable dual-RNA-guided DNA endonuclease in adaptive bacterial immunity. Science. 2012; 337:816-21. The terms crRNA and tracrRNA are used interchangeably with the terms tracr-mate RNA and tracr RNA herein. Since the discovery of the CRISPR-Cas9 system single polynucleotide guide RNAs have been developed and successfully applied just as effectively as the natural two part guide RNA complex.

Donor DNA: The term “donor DNA” means a polynucleotide that comprises a nucleotide sequence of interest for modifying a target site in the genome of a fungal cell. The donor DNA can be double-stranded DNA. The nucleotide sequence of the donor DNA can be any nucleotide sequence such as a gene or a region of a gene, one or more nucleotides for introducing a mutation into a gene, a gene disruption sequence, etc. In one aspect, the donor DNA further comprises a first region of homology and a second region of homology to corresponding regions of the target site for incorporation of the donor DNA into the double-strand break by homologous recombination, i.e., the donor DNA has a high degree of homology to the sequence immediately upstream and downstream of the intended editing site. The term “donor DNA” is also understood herein to mean “DNA repair template”.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Codon-optimized gene: The term “codon-optimized gene” means a gene having its frequency of codon usage optimized to the frequency of preferred codon usage of a host cell. The nucleic acid changes made to codon-optimize a gene do not change the amino acid sequence of the encoded polypeptide of the parent gene.

Control sequences: The term “control sequences” means nucleic acid sequences necessary for expression of a polynucleotide comprising a non-coding RNA or a polynucleotide encoding a polypeptide. Each control sequence may be native (i.e., from the same gene) or heterologous (i.e., from a different gene) to the polynucleotide encoding the polypeptide or native or heterologous to each other. Such control sequences include, but are not limited to, a leader, polyadenylation sequence, propeptide sequence, promoter, signal peptide sequence, and transcription terminator. At a minimum, the control sequences include a promoter and a transcriptional stop signal. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion. The term “expression” also means production of a non-coding RNA (e.g., a single guide RNA).

Expression vector: The term “expression vector” means a linear or circular DNA molecule that comprises a polynucleotide encoding a polypeptide or a non-coding polynucleotide (e.g., a single guide RNA) and is operably linked to control sequences that provide for its expression.

Genome: The term “genome” means the complete set of genetic information in a fungal cell which is present as long molecules of DNA called chromosomes and extrachromosomal elements of DNA (e.g., plasmids) and RNA.

Guide RNA or single guide RNA: The term “guide RNA” (gRNA) or “single guide RNA” (sgRNA) means an engineered single-stranded RNA, involving (1) the targeting function of the CRISPR RNA (crRNA) sequence (for MAD7 and Cpf1), or (2) the targeting function of the CRISPR RNA (crRNA) and the nuclease-binding function of the transactivating CRISPR RNA (tracrRNA) sequence (for Cas9). For the Cas9 endonuclease, the crRNA sequence is an approximately 20 nucleotide sequence that defines the genomic target of interest for modification via homology and directs Cas9 endonuclease activity. The 20 nucleotide sequence acts as a “guide”, which recruits the Cas9/gRNA complex to a specific DNA target site based on the crRNA sequence, directly upstream of a protospacer adjacent motif (PAM), through RNA-DNA base pairing. The PAM is required for cleavage, but is not part of the gRNA or sgRNA sequence. The Cas9 endonuclease will cleave approximately 3 bases upstream of the PAM. For the MAD7 and Cpf1 endonucleases, they are guided by a single CRISPR RNA (crRNA) and does not require a transactivating CRISPR RNA (tracrRNA). The MAD7 and Cpf1 endonucleases cleave DNA distal to its PAM after the +18/+23 position of the protospacer creating a staggered DNA overhang.

Homologous recombination: The term “homologous recombination” means the exchange of DNA fragments between two DNA molecules at sites of homology via a classical Campbell-type homologous recombination event.

Host cell: The term “host cell” means any cell type that is susceptible to transformation, transfection, transduction, or the like with a nucleic acid construct or expression vector comprising a polynucleotide. The term “host cell” encompasses any progeny of a parent cell that is not identical to the parent cell due to mutations that occur during replication.

Mutant: The term “mutant” means a polynucleotide comprising an alteration, i.e., a substitution, an insertion, and/or a deletion, at one or more (e.g., several) positions. A substitution means replacement of the nucleotide occupying a position with a different nucleotide; a deletion means removal of the nucleotide occupying a position; and an insertion means adding a nucleotide adjacent to and immediately following a nucleotide occupying a position.

Nucleic acid construct: The term “nucleic acid construct” means a nucleic acid or polynucleotide molecule, either single- or double-stranded, which is isolated from a naturally occurring gene or is modified to contain segments of nucleic acids in a manner that would not otherwise exist in nature or which is synthetic, which may comprise one or more control sequences.

Operably linked: The term “operably linked” means a configuration in which a control sequence is placed at an appropriate position relative to a polynucleotide such that the control sequence directs expression of the polynucleotide.

Promoter: The term “promoter” means a DNA sequence that defines where transcription of a gene by an RNA polymerase begins. A promoter is located directly upstream or at the 5′ end of the transcription start site of a gene. RNA polymerase and the necessary transcription factors bind to the promoter sequence and initiate transcription.

Sequence identity: The relatedness between two amino acid sequences or between two nucleotide sequences is described by the parameter “sequence identity”.

For purposes of the present disclosure, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present disclosure, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Transcriptional terminator: The term “transcriptional terminator” means a DNA sequence downstream of the polynucleotide sequence of a gene which is recognized by RNA polymerase as a signal to stop synthesizing and release nascent RNA from the transcriptional complex.

Transfer RNA: The term “transfer RNA” means a molecule composed of RNA, typically 73 to 94 nucleotides in length, that serves as the physical link between the nucleotide sequence of nucleic acids and the amino acid sequence of proteins. Transfer RNA carries an amino acid to the protein synthetic machinery of a cell (ribosome) as directed by a three-nucleotide sequence (codon) in a messenger RNA (mRNA) and attaches the correct amino add to a protein chain that is being synthesized at the ribosome cell when the anticodon of the tRNA pairs with a codon on the mRNA being translated into the protein. There are at least 20 species of transfer RNA, each species capable of combining with a specific amino acid. Each type of transfer RNA molecule can be attached to only one type of amino acid, so each organism has many types of transfer RNA. Since the genetic code contains multiple codons that specify the same amino acid, there are many transfer RNA molecules bearing different anticodons which also carry the same amino acid. There are often multiple species of tRNA for each codon and as a result there can be more than one hundred tRNA genes within the genome of a particular fungal cell. For example, see Hani and Feldman, 1998, Nucleic Acids Res. 26: 689-696. The terms “transfer RNA” and “tRNA” are used interchangeably herein.

U6 promoter: The term “U6 promoter” means a promoter obtained from a U6 small nuclear RNA (snRNA) gene and transcribed by RNA polymerase III.

RNA polymerase III: The term “RNA polymerase III” means a nucleotidyl transferase that polymerizes ribonucleotides using DNA genes as templates (Paule and White, 2000, Nucleic Acids Res. 28(6):1283) to produce small ribonucleic (RNA) molecules including, but not limited to, aminoacyl transfer RNAs, 5S ribosomal RNAs, splicecomal RNAs (snRNAs), and U6 small nuclear RNAs.

### DETAILED DESCRIPTION OF THE INVENTION

In a first aspect, the invention relates to methods for introducing one or more desired nucleotide modification(s) in at least one target sequence in the genome of a microorganism cell using a polynucleotide-guided endonuclease, said method comprising the steps of:
a) providing a microorganism host cell comprising at least one genome target sequence to be modified located in the vicinity of a protospacer adjacent motif (PAM) sequence for the polynucleotide-guided endonuclease;
b) transforming the microorganism host cell with:
   i) the polynucleotide-guided endonuclease and at least one suitable guide polynucleotide for the at least one target sequence to be modified, OR one or more polynucleotide encoding the polynucleotide-guided endonuclease and encoding at least one suitable guide polynucleotide for the at least one target sequence to be modified, and
   ii) at least one single-stranded oligonucleotide capable of hybridizing with the at least one genome target sequence, said oligonucleotide comprising the one or more desired nucleotide modification(s);

wherein the polynucleotide-guided endonuclease interacts with the guide polynucleotide and with the genome and cuts or nicks the genome, and wherein the at least one single-stranded nucleotide directs DNA repair across the cut or nick, thereby introducing the one or more desired modification(s) into the target sequence of the genome with an efficiency of at least:

70% when the cut or nick is located 10-20 nucleotides from the desired nucleotide modification(s); preferably at least 75%, 80%, or 85% when the cut or nick is located 10-20 nucleotides from the desired nucleotide modification(s); most preferably at least 90% when the cut or nick is located 10-20 nucleotides from the desired nucleotide modification(s);

60% when the cut or nick is located 21-30 nucleotides from the desired nucleotide modification(s); preferably at least 65% when the cut or nick is located 21-30 nucleotides from the desired nucleotide modification(s); more preferably at least 70% when the cut or nick is located 21-30 nucleotides from the desired nucleotide modification(s);

50% when the cut or nick is located 31-43 nucleotides from the desired nucleotide modification(s); preferably at least 55% when the cut or nick is located 31-43 nucleotides from the desired nucleotide modification(s); more preferably at least 60% when the cut or nick is located 31-43 nucleotides from the desired nucleotide modification(s);

40% when the cut or nick is located 44-52 nucleotides from the desired nucleotide modification(s); preferably at least 45% when the cut or nick is located 44-52 nucleotides from the desired nucleotide modification(s); more preferably at least 50% when the cut or nick is located 44-52 nucleotides from the desired nucleotide modification(s); or

30% when the cut or nick is located at least 53 nucleotides from the desired nucleotide modification(s); preferably at least 35% when the cut or nick is located at least 53 nucleotides from the desired nucleotide modification(s); more preferably at least 40% when the cut or nick is located at least 53 nucleotides from the desired nucleotide modification(s).

### Host Cells

The present invention also relates to microorganism host cells. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptococcus. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptococcus cell including, but not limited to, Streptococcus achromogenes, Streptococcus avermitilis, Streptococcus coelicolor, Streptococcus griseus, and Streptococcus lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptococcus cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

It is advantageous in the methods of the present invention to employ a filamentous fungal host cell that is unable to quickly repair the nicked or cut target sequence(s) without integration of the modified donor part of the genome.

Accordingly, it is preferred that the filamentous fungal host cell provided in step (A) of the first aspect of the invention comprises an inactivated non-homologous end joining (NHEJ) system; preferably the cell comprises an inactivated DNA Ligase D (LigD) and/or DNA-end-binding protein Ku; even more preferably the cell comprises inactivated ligD, ku70 and or ku80 gene or homologoue(s) thereof.

In a preferred embodiment, the microorganism host cell is transformed with a polynucleotide encoding a polypeptide of interest either before or after the steps in the first aspect; preferably the polypeptide of interest is an enzyme; preferably the enzyme is a hydrolase, isomerase, ligase, lyase, oxidoreductase, or transferase; even more preferably the enzyme is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

### Polynucleotide-Guided Endonucleases

In the methods of the present disclosure, any polynucleotide-guided endonuclease can be used, both RNA- and DNA-guided endonucleases are contemplated.

The RNA-guided DNA endonuclease can be a Cas endonuclease, a Mad endonuclease, or a Cpf endonuclease.

In one aspect, the Cas endonuclease can be any Cas endonuclease or a functional fragment thereof useful in the methods of the present disclosure. In one embodiment, the Cas endonuclease is a Cas9 endonuclease. Examples of Cas9 endonucleases are the Cas9 endonucleases from the following bacterial species: Streptococcus sp. (e.g., S. pyogenes, S. mutans, and S. thermophilus), Campylobacter sp. (e.g., C. jejuni), Neisseria sp. (e.g., N. meningitidis), Francisella sp. (e.g., F. novicida), and Pasteurella sp. (e.g., P. multocida). For a discussion of Cas9 endonucleases, see Makarova et al., 2015, Nature 13: 722-736.

In another embodiment, the Cas9 endonuclease is a Streptococcus pyogenes Cas9 or homologue thereof. In another embodiment, the Cas9 endonuclease is a Streptococcus mutans Cas9 endonuclease. In another embodiment, the Cas9 endonuclease is a Streptococcus thermophilus Cas9 endonuclease. In another embodiment, the Cas9 endonuclease is a Campylobacter jejuni Cas9 endonuclease. In another embodiment, the Cas9 endonuclease is a Neisseria meningitidis Cas9 endonuclease. In another embodiment, the Cas9 endonuclease is a Francisella novicida Cas9 endonuclease. In another embodiment, the Cas9 endonuclease is a Pasteurella multocida Cas9 endonuclease.

In another embodiment, the Cas9 endonuclease variant has only one active nuclease domain. In a more preferred embodiment, the Cas9 endonuclease variant comprises a substitution with alanine in the amino acid position corresponding to position 10 of the Streptomyces pyogenes Cas9 amino acid sequence. In a most preferred embodiment, the polynucleotide-guided endonuclease has only one active nuclease domain; preferably said variant is a Streptococcus pyogenes Cas9 comprising a substitution of aspartic acid for alanine in the amino acid position corresponding to position 10, D10A

In another embodiment, the Cas9 endonuclease is a variant of a parent Cas9 endonuclease. In one embodiment, the Cas9 endonuclease variant is a Cas9 nickase in which the RuvC domain is inactivated by a D10A mutation in the Cas9 endonuclease from Streptococcus pyogenes (Sander and Joung, 2013, Nature Biotechnology 1-9). It is expected that other Class-II Cas9 enzymes may be modified similarly.

In another aspect, the Mad endonuclease can be any Mad endonuclease or a functional fragment thereof useful in the methods of the present disclosure. In one embodiment, the Mad endonuclease is a MAD7 endonuclease. An example of a MAD7 endonuclease is the MAD7 endonuclease from Eubacterium rectale. For a discussion of the MAD7 endonuclease, see WO 2018/071672.

In another embodiment, the MAD7 endonuclease is a Eubacterium MAD7 endonuclease. In another embodiment, the Eubacterium MAD7 endonuclease is an Eubacterium rectale MAD7 endonuclease.

In one aspect, the Cpf endonuclease can be any Cpf endonuclease or a functional fragment thereof useful in the methods of the present disclosure. In one embodiment, the Mad endonuclease is a Cpf1 endonuclease. Examples of Cpf1 endonucleases are the Cpf1 endonucleases from Acidaminococcus sp., Lachnospiraceae sp., and Francisella novicide. For a discussion of the Cpf1 endonuclease, see Zetsche et al., 2015, Cell 163(3) 759-771.

In another embodiment, the Cpf1 endonuclease is an Acidaminococcus Cpf1 endonuclease. In another embodiment, the Cpf1 endonuclease is a Lachnospiraceae Cpf1 endonuclease. In another embodiment, the Cpf1 endonuclease is a Francisella Cpf1 endonuclease. In another embodiment, the Cpf1 endonuclease is a Francisella novicide Cpf1 endonuclease.

In another embodiment, a gene encoding the RNA-guided DNA endonuclease is a codon-optimized synthetic sequence for expression in a fungal cell.

In another embodiment, the RNA-guided DNA endonuclease gene is operably linked to one or more polynucleotides encoding nuclear localization signals so the expressed endonuclease is efficiently transported from the cytoplasm to the nucleus. Examples of nuclear localization signals are the SV40 nuclear localization signal, Aspergillus nidulans GATA transcription factor (AreA), Trichoderma reesei transcriptional regulator for cellulase and hemicellulase gene expression (XYR1), Trichoderma reesei blue light regulator 2 (blr2), Xenopus laevis oocyte Nucleoplasmin nuclear localization signal, Caenorhabditis elegans transcription factor EGL-13 nuclear localization signal, Homo sapiens transcription factor c-Myc nuclear localization signal, and Escherichia coli replication fork arresting protein (TUS-protein) nuclear localization signal.

### Guide RNA

The guide RNA (gRNA) in CRISPR-Cas9 genome editing constitutes the re-programmable part that makes the system so versatile. In the natural Streptomyces pyogenes system, the guide RNA is a complex of two RNA polynucleotides, a crRNA containing about 20 nucleotides that determine the specificity of the Cas9 enzyme and a tracrRNA which hybridizes to the crRNA to form an RNA complex that interacts with the Cas9 endonuclease. See Jinek et al., 2012, Science 337: 816-821.

Since the discovery of the CRISPR-Cas9 system single guide RNAs have been developed and successfully applied just as effectively as the natural two-part guide RNA complex.

In the methods of the present disclosure, any guide RNA system can be used.

In one embodiment, the guide RNA is the natural Streptomyces pyogenes system (Jinek et al., 2012, Science 337(6096): 816-821).

In another embodiment, the guide RNA, known as a single guide RNA (sgRNA), is an engineered single-stranded chimeric RNA, which combines the scaffolding function of the bacterial transactivating CRISPR RNA (tracrRNA) with the specificity of the bacterial CRISPR RNA (crRNA). The last 17-20 bp at the 5′ end of the crRNA acts as a “guide”, which recruits the Cas9/gRNA complex to a specific DNA target site, directly upstream of a protospacer adjacent motif (PAM), through RNA-DNA base pairing.

In another embodiment, the single guide RNA comprises a first RNA comprising 17 to 20 or more nucleotides that are at least 85%, e.g., 90%, 95%, 96%, 97%, 98%, 99% or 100%, complementary to and capable of hybridizing to the target sequence.

In another embodiment, the first RNA comprising the 17 to 20 or more nucleotides are at least 90%, 95%, 97%, 98%, 99% or even 100% complementary to and capable of hybridizing to the target sequence.

In another embodiment, the single guide RNA is a Streptomyces pyogenes Cas9 guide RNA. In another embodiment, the guide RNA is an Eubacterium rectale MAD7 guide RNA. In another embodiment, the guide RNA is a Cpf1 guide RNA.

### Genome Target Sequence

At least one genome target sequence is to be modified by the methods of the invention and the target sequence must be located in the vicinity of a protospacer adjacent motif (PAM) sequence for the polynucleotide-guided endonuclease; preferably the at least one genome target sequence to be modified is located from 10 to 1,000 nucleotides away from the protospacer adjacent motif (PAM) sequence for the polynucleotide-guided endonuclease in the genome of the microorganism host cell; preferably the at least one genome target sequence to be modified is located from 10 to 500 nucleotides away from the protospacer adjacent motif (PAM) sequence for the polynucleotide-guided endonuclease in the genome of the microorganism host cell; more preferably the at least one genome target sequence to be modified is located from 20 to 250 nucleotides away from the protospacer adjacent motif (PAM) sequence for the polynucleotide-guided endonuclease in the genome of the microorganism host cell; even more preferably the at least one genome target sequence to be modified is located from 21 to 100 nucleotides away from the protospacer adjacent motif (PAM) sequence for the polynucleotide-guided endonuclease in the genome of the microorganism host cell; even more preferably the at least one genome target sequence to be modified is located from 22 to 100 nucleotides away from the protospacer adjacent motif (PAM) sequence for the polynucleotide-guided endonuclease in the genome of the microorganism host cell; even more preferably the at least one genome target sequence to be modified is located from 23 to 100 nucleotides away from the protospacer adjacent motif (PAM) sequence for the polynucleotide-guided endonuclease in the genome of the microorganism host cell; even more preferably the at least one genome target sequence to be modified is located from 24 to 100 nucleotides away from the protospacer adjacent motif (PAM) sequence for the polynucleotide-guided endonuclease in the genome of the microorganism host cell; even more preferably the at least one genome target sequence to be modified is located from 25 to 100 nucleotides away from the protospacer adjacent motif (PAM) sequence for the polynucleotide-guided endonuclease in the genome of the microorganism host cell; even more preferably the at least one genome target sequence to be modified is located from 26 to 100 nucleotides away from the protospacer adjacent motif (PAM) sequence for the polynucleotide-guided endonuclease in the genome of the microorganism host cell; even more preferably the at least one genome target sequence to be modified is located from 27 to 100 nucleotides away from the protospacer adjacent motif (PAM) sequence for the polynucleotide-guided endonuclease in the genome of the microorganism host cell; even more preferably the at least one genome target sequence to be modified is located from 28 to 100 nucleotides away from the protospacer adjacent motif (PAM) sequence for the polynucleotide-guided endonuclease in the genome of the microorganism host cell; even more preferably the at least one genome target sequence to be modified is located from 29 to 100 nucleotides away from the protospacer adjacent motif (PAM) sequence for the polynucleotide-guided endonuclease in the genome of the microorganism host cell; even more preferably the at least one genome target sequence to be modified is located from 30 to 100 nucleotides away from the protospacer adjacent motif (PAM) sequence for the polynucleotide-guided endonuclease in the genome of the microorganism host cell.

The actual cut or nick in the genome target sequence is made within a “protospacer-complementary” sequence located immediately next to the PAM sequence in the genome. The protospacer-complementary sequence is usually 20 nucleotides in length or so, in order to allow its hybridization to the corresponding protospacer sequence of the guide polynucleotide, but even shorter sequences have been shown to work, such as, a 17 nucleotide protospacer in the guide and corresponding protospacer-complementary 17 nucleotide sequence in the genome. The at least one genome target sequence to be modified may be located anywhere in the genome but will often be within a coding sequence or open reading frame.

In a preferred embodiment, at least two genome target sequences in the host cell are modified by at least one insertion, deletion and/or substitution of one or more nucleotide or codon. In another preferred embodiment, the one or more desired nucleotide modification(s) comprises at least one insertion, deletion and/or substitution of one or more nucleotide or codon.

Each protospacer-complementary sequence in the genome needs to have a suitable protospacer adjacent motif (PAM) located next to it to allow the corresponding polynucleotide-guided endonuclease to bind and cut or nick the genome. The term “protospacer adjacent motif” or “PAM” means a 2-6 base pair DNA sequence immediately downstream or upstream of the target site in the genome, which is recognized directly by an RNA-guided DNA endonuclease, e.g., a Cas9, MAD7, or Cpf1 endonuclease, to promote cleavage of the target site by the RNA-guided DNA endonuclease. The Cas9 endonuclease from Streptococcus pyogenes recognizes 5′-NGG on the 3′ end of the gRNA sequence. The MAD7 endonuclease from Eubacterium rectale recognizes 5′-TTTV on the 5′ end of the gRNA sequence, but 5′-YTTV and YTTN also work to some extent. The Cpf1 endonuclease from Acidaminococcus sp. and Lachnospiraceae sp. recognize 5′-TTTN and the Cpf1 endonuclease Francisella novicide recognizes 5′-TTN-3′ on the 5′ end of the gRNA. For an overview of other PAM sequences, see, for example, Shah, S. A. et al, Protospacer recognition motifs, RNA Biol. 2013 May 1; 10(5): 891-899.

### Single-Stranded Oligonucleotide

The single-stranded oligonucleotide of the first aspect of this invention serves as donor DNA, also known as a DNA repair template. The single-stranded oligonucleotide comprises a nucleotide sequence for modifying or editing a target site of a microorganism host cell, and additional homologous sequence corresponding to immediately upstream and downstream of the target site (termed “5′ homology sequence” and “3′ homology sequence”). The length of each homology sequence may be varied, but in general the single-stranded oligonucleotide comprises at least 15 unmodified nucleotides on the opposite side of the cut or nick in the genome relative to the modification(s) and at least 15 unmodified nucleotides on the opposite side of the modification(s) relative to the cut or nick in the genome; preferably at least 16 unmodified nucleotides; preferably at least 17, 18, 19, 20, 21, 22, 23, 24, 25, 26 or at least 27 unmodified nucleotides on each side.

Non-limiting examples for modifying a target site are deleting a gene or a portion thereof, disrupting a gene, altering a nucleotide or nucleotides within a gene, replacing a gene with a heterologous gene encoding a protein with improved biological activity, e.g., a homolog or variant, introducing a mutation into a gene, replacing a gene with a heterologous gene encoding a protein with different biological activity, inserting a gene, or repairing a gene.

In an embodiment, the nucleotide sequence of interest for modifying the target site comprises at least 30, 40, 50, 60, 70, 80, 90, 100, 150, 200, 250, 300, 350, 400, 450, 500, 550, 600, 650, 700, 750, 800, 850, 900, 950, 1,000, 2,000, 4,000, 6,000, 8,000, or 10,000 nucleotides.

It may be advantageous to include one or more additional mutations in the PAM- or protospacer-corresponding sequences of the single-stranded oligonucleotide(s) so that, when the method of the first aspect has worked as intended and the desired modifications have been introduced into the genome, the PAM site and the protospacer-complementary sequences in the genome will have been mutated to avoid any subsequent cut or nick from residual activity of the polynucleotide-guided endonuclease.

Accordingly, in a preferred embodiment, the at least one single-stranded oligonucleotide in addition to the one or more desired nucleotide modification(s) also comprises one or more mutation in the PAM or protospacer sequence, wherein said one or more mutation effectively blocks the polynucleotide-guided endonuclease when introduced into the target sequence.

### Polynucleotide for Polypeptide Expression

In one embodiment, the nucleotide sequence of interest is a gene. The gene of interest can be. an oxidoreductase, a transferase, a hydrolase, a lyase, an isomerase, or a ligase. In another aspect, the polypeptide is an acetylmannan esterase, acetylxylan esterase, aminopeptidase, alpha-amylase, arabinanase, arabinofuranosidase, carbohydrase, carboxypeptidase, catalase, cellobiohydrolase, cellulase, chitinase, coumaric acid esterase, cyclodextrin glycosyltransferase, cutinase, cyclodextrin glycosyltransferase, deamidase, deoxyribonuclease, dispersin, endoglucanase, esterase, feruloyl esterase, GH61 polypeptide having cellulolytic enhancing activity, alpha-galactosidase, beta-galactosidase, glucocerebrosidase, glucose oxidase, alpha-glucosidase, beta-glucosidase, glucuronidase, glucuronoyl esterase, haloperoxidase, hemicellulase, invertase, isomerase, laccase, ligase, lipase, lysozyme, mannanase, mannosidase, mutanase, oxidase, pectinolytic enzyme, peroxidase, phosphodiesterase, phospholipase, phytase, phenoloxidase, polyphenoloxidase, proteolytic enzyme, ribonuclease, alpha-1,6-transglucosidase, transglutaminase, urokinase, xanthanase, xylanase, or beta-xylosidase

In another aspect, the nucleotide sequence of interest is a region of a gene.

The region can be, for example, an open reading frame, a protein coding sequence, an intron site, an intron enhancing motif, a mRNA splice site, a promoter, a transcriptional regulatory element, a transcriptional terminator, and a translational regulatory element.

Techniques used to isolate or clone a gene as the nucleotide sequence of interest are known in the art and include isolation from genomic DNA or cDNA, or a combination thereof. The cloning of the gene from genomic DNA can be affected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

Any gene that encodes, for example, a polypeptide may be modified at the nucleotide sequence level to serve as the nucleotide sequence of interest. Such modifications may not alter the amino acid sequence of the encoded polypeptide or they may lead to changes in the amino acid sequence, such as, deletions, insertions, or substitutions.

If an amino acid is substituted with another amino acid with similar characteristics it may be termed a conservative substitution. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes can be of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

### Endonuclease or Guide-Polynucleotide Expression

The methods of the present disclosure relate to several nucleic acid constructs that are used for modifying a target site in the genome of a fungal cell.

In one aspect, the nucleic acid construct comprises a polynucleotide encoding an polynucleotide-guided endonuclease, e.g., a Cas9, or MAD7 endonuclease, for introducing a double-stranded cut or single-stranded cut (nick) at a target site in the genome of a fungal cell, wherein the fungal cell comprises a protospacer adjacent motif sequence for the RNA-guided DNA endonuclease immediately in the vicinity of the target site.

In another aspect, the nucleic acid construct comprises (a) a U6 promoter sequence operably linked at the 5′ end of (1) a sequence encoding a transfer RNA and (2) a sequence encoding a single guide RNA at the 3′ end of the transfer RNA sequence, and (b) a U6 transcriptional terminator sequence operably linked at the 3′ end of the sequence encoding the single guide RNA, wherein the single guide RNA directs the RNA-guided DNA endonuclease, e.g., a Cas9, MAD7, or Cpf1 endonuclease, to a target site in the genome of a fungal cell to introduce a double-strand break, and wherein the nucleic acid construct increases the frequency of the RNA-guided DNA endonuclease in producing the double-strand break at the target site.

In a preferred embodiment, the nucleic acid construct comprising the polynucleotide encoding the RNA-guided DNA endonuclease, e.g., the Cas9, MAD7, or Cpf1 endonuclease, and the nucleic acid construct comprising (a) a U6 promoter sequence operably linked at the 5′ end of (1) a sequence encoding a transfer RNA, (2) a sequence encoding a single guide RNA at the 3′ end of the transfer RNA sequence, and (b) a U6 transcriptional terminator sequence operably linked at the 3′ end of the sequence encoding the single guide RNA are on a single DNA fragment or a single vector.

### Nucleic Acid Constructs for Polypeptide Expression

The present invention also relates to nucleic acid constructs that are transformed into the filamentous fungal host cell for polypeptide expression.

A polynucleotide to be expressed is operably linked to one or more control sequences that direct the expression of the coding sequence in a suitable host cell under conditions compatible with the control sequences. The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptococcus coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCI B 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptococcus hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as from around 30 to around 10,000 base pairs, or from around 400 to around 10,000 base pairs, or from around 800 to around 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Reducing or Eliminating Gene Expression

Reducing or eliminating expression of a polynucleotide using, for example, one or more nucleotide insertion, disruption, substitution or deletion, is well known in the art.

In a method of the first aspect of invention, in a preferred embodiment, the genome of the host cell is modified to ensure that expression of a polynucleotide is reduced or eliminated, for example by modification, inactivation or full/partial deletion. The polynucleotide to be modified, inactivated or deleted may be, for example, the coding region or a part thereof essential for activity, or a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

An example of a convenient way to eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The polypeptide-deficient mutant cells are particularly useful as host cells for expression of native and heterologous polypeptides. Therefore, the present invention further relates to methods of producing a native or heterologous polypeptide, comprising (a) cultivating the mutant cell under conditions conducive for production of the polypeptide; and (b) recovering the polypeptide. The term “heterologous polypeptides” means polypeptides that are not native to the host cell, e.g., a variant of a native protein. The host cell may comprise more than one copy of a polynucleotide encoding the native or heterologous polypeptide.

The present invention is further described by the following examples that should not be construed as limiting the scope of the invention.

### EXAMPLES

The purpose of these examples is to show that directed mutagenesis or genome editing using a polynucleotide-guided endonuclease, such as, Cas9 or MAD7, is possible, where a single-stranded oligonucleotide is used as donor DNA.

### Strains

Trichoderma reesei BTR213 described in WO 2013/086633. Trichoderma reesei strain TrGMEr62-24a2-1 is a ku70 disrupted strain of T. reesei BTR213.

Aspergillus oryzae AT526 is a ligD disrupted strain derived from JaL1903, which is described in WO18167153 (example 4).

### Media and Solutions

LB+Amp medium was composed of 10 g of Bacto™ tryptone, 5 g of Bacto™ yeast extract, 5 g of sodium chloride, 1 ml of ampicillin at 100 mg/ml (filter sterilized and added after autoclaving), and deionized water to 1 liter. The solution was sterilized by autoclaving.

PDA plates were composed of 39 g of Difco™ potato dextrose agar and deionized water to 1 liter. The solution was sterilized by autoclaving.

PDA+1 M sucrose plates were composed of 39 g of Difco™ potato dextrose agar, 342.30 g sucrose and deionized water to 1 liter. The solution was sterilized by autoclaving.

PEG buffer was composed of 50% polyethylene glycol (PEG) 4000, 10 mM Tris-HCl pH 7.5, and 10 mM CaCl₂) in deionized water. The solution was filter sterilized.

STC was composed of 1 M sorbitol, 10 mM Tris pH 7.5, and 50 mM CaCl₂ in deionized water. The solution was filter sterilized.

TBE buffer was composed of 10.8 g of Tris Base, 5 g of boric acid, 4 ml of 0.5 M EDTA pH 8, and deionized water to 1 liter.

TE buffer was composed of 1 M Tris pH 8.0 and 0.5 M EDTA pH 8.0.

2×YT+Amp plates were composed of 16 g of Bacto™ tryptone, 10 g of Bacto™ yeast extract, 5 g of NaCl, 15 g of Bacto™ agar, 1 ml of ampicillin at 100 mg/ml (filter sterilized and added after autoclaving), and deionized water to 1 liter. The solution was sterilized by autoclaving.

YP medium was composed of 1% Bacto™ yeast extract and 2% Bacto™ peptone in deionized water. The solution was sterilized by autoclaving.

YPD medium was composed of 1% Bacto™ yeast extract, 2% Bacto™ peptone and 2% glucose. The solution was sterilized by autoclaving.

Top agar solution was composed of 0.18 μM Na₂B₄O₇, 2.3 μM CuSO₄, 4.7 μM FeSO₄, 4.7 μM MnSO₄, 3.6 μM Na₂MoO₄, 45 μM ZnSO₄, 7 mM KCl, 4.3 mM MgSO₄, 1.2 mM KH₂PO₄, 1 M sucrose, 5 ml Tris-HCl (1M, pH 7.5) and 10 g of SeqPlaque GTG agarose in deionized water (1 liter final volume). The solution was sterilized by autoclaving.

Medium for sucrose+urea plates was composed of 0.18 μM Na₂B₄O₇, 2.3 μM CuSO₄, 4.7 μM FeSO₄, 4.7 μM MnSO₄, 3.6 μM Na₂MoO₄, 45 μM ZnSO₄, 7 mM KCl, 4.3 mM MgSO₄, 1.2 mM KH₂PO₄, 1 M sucrose and 20 g of Bacto™ agar in deionized water (1 liter final volume). The solution was sterilized by autoclaving. After autoclaving, urea (1 M, sterile filtered) was added to a final concentration of 10 mM.

Medium for sucrose+urea+Triton plates was composed of 0.18 μM Na₂B₄O₇, 2.3 μM CuSO₄, 4.7 μM FeSO₄, 4.7 μM MnSO₄, 3.6 μM Na₂MoO₄, 45 μM ZnSO₄, 7 mM KCl, 4.3 mM MgSO₄, 1.2 mM KH₂PO₄, 1 M sucrose and 20 g of Bacto™ agar in deionized water (1 liter final volume). The solution was sterilized by autoclaving. After autoclaving, urea (1 M, sterile filtered) was added to a final concentration of 10 mM and a few drops of Triton X-100 (sterilized by autoclaving) was added.

### Example 1: Trichoderma reesei Protoplast Generation

Protoplast preparation and transformation of Trichoderma reesei were performed using a protocol similar to Penttila et al., 1987, Gene 61: 155-164. Briefly, T. reesei was cultivated in two shake flasks, each containing 25 ml of YPD medium, at 27° C. for 17 hours with gentle agitation at 90 rpm. Mycelia were collected by filtration using a Vacuum Driven Disposable Filtration System (Millipore) and washed twice with deionized water and twice with 1.2 M sorbitol. Protoplasts were generated by suspending the washed mycelia in 30 ml of 1.2 M sorbitol containing 5 mg/ml of Yatalase™ (Takara Bio USA, Inc.) and 0.5 mg/ml of Chitinase (Sigma Chemical Co.) 60-75 minutes at 34° C. with gentle shaking at 75-90 rpm. Protoplasts were collected by centrifugation at 834×g for 6 minutes and washed twice with cold 1.2 M sorbitol. The protoplasts were counted using a hemocytometer and re-suspended to a final concentration of 1×10⁸ protoplasts per ml of STC. Aliquots (1.1 ml) of the protoplast solution were placed in a Mr. Frosty™ freezing container (Thermo Fisher Scientific) prepared according to the manufacturer's instructions and placed at −80° C. for later use.

### Example 2: CRISPR/Cas9 Backbone Vector pSMAI290

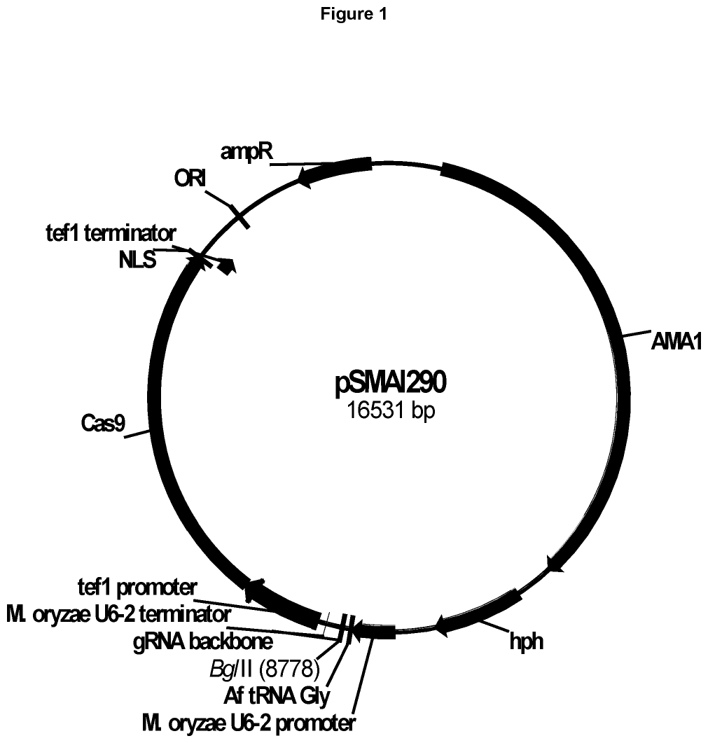

Plasmid pSMAI290 (SEQ ID NO:1, FIG. 1) is a CRISPR/Cas9 expression plasmid used to clone in protospacers into BgIII digested pSMAI290 using an NEBuilder® HiFi DNA Assembly Cloning Kit (New England Biolabs Inc.). Plasmid pSMAI290 contains a Streptococcus pyogenes Cas9 protein coding sequence (nucleotides 9968-14,098 in pSMAI290) and codon-optimized for use in Aspergillus niger and a SV40 nuclear localization signal (NLS; nucleotides 14,072-14,095) at the 3′ end of the S. pyogenes Cas9 open reading frame to ensure the Cas9 would be localized to the nucleus. The expression of the S. pyogenes Cas9 is under control of the Aspergillus nidulans tef1 promoter (nucleotides 9082-9967) and terminator (nucleotides 14,099-14,297) from pFC330-333 (Nødvig et al., 2015, PLoS One 10(7): 1-18). Plasmid pSMAI290 also has all the elements for single guide RNA (sgRNA) expression, which consists of the Magnaporthe oryzae U6-2 promoter (nucleotides 8186-8685), Aspergillus fumigatus tRNAgly(GCC)1-6 sequence with the region downstream the structural tRNA removed (nucleotides 8686-8776), BgIII restriction enzyme recognition sequence (nucleotides 8777-8782), S. pyogenes single guide RNA sequence (nucleotides 8783-8860), and M. oryzae U6-2 terminator (nucleotides 8861-9075). For selection in T. reesei, plasmid pSMAI290 contains the hygromycin phosphotransferase gene from pHT1 (Cummings et al., 1999, Curr. Genet. 36: 371) (nucleotides 6712-7743), conferring resistance to hygromycin B, and the autonomous maintenance in Aspergillus (AMA1) sequence (Gems et al., 1991, Gene 98: 61-67) (nucleotides 569-6293) for extrachromosomal replication of pSMAI290 in T. reesei. The single guide RNA and the Cas9-SV40 NLS expression elements in pSMAI290 were confirmed by DNA sequencing with a Model 377 XL Automated DNA Sequencer using dye-terminator chemistry.

### Example 3: Construction of pNJ00502-504

Plasmid vector preparation. Plasmid pSMAI290 was digested with the restriction enzyme BgIII (Anza™ 19 BgIII, Thermo Fisher Scientific). The restriction reaction contained: 5 μg of pSMAI290 plasmid DNA, 1× Anza™ buffer, 50 units of BgIII, and sterile Milli-Q water up to 50 μl final volume. The reaction was incubated at 37° C. for 1 hour. Following restriction enzyme digestion, the digest was subjected to 0.7% agarose gel electrophoresis in TBE buffer and the band representing the digested pSMAI290 was excised from the gel and purified using a NucleoSpin® Gel and PCR Clean-up kit (Macherey-Nagel) according to the manufacturer's instructions.

Protospacer design. Three different twenty base-pairs protospacers were designed for the ACE3 locus (SEQ ID NO:2) to direct the Cas9 enzyme to the target site and create a double stranded break. Protospacers were selected by finding an appropriate protospacer adjacent motif (PAM) with the sequence NGG, where N represents any nucleotide (A, C, G, or T). Once an appropriate PAM site was identified, the twenty base-pairs immediately adjacent to the 5′ side of the PAM site were selected as the protospacer. Protospacers that contained more than two contiguous T nucleotides were rejected to avoid possible stuttering of RNA polymerase.

Each protospacer with its extension sequences used for cloning (oNJ336, oNJ338 and oNJ340) was synthesized as a single-stranded oligonucleotide by Thermo Fisher Scientific, Inc. All protospacer oligonucleotides were diluted to a final working concentration of 1 μM:

oNJ336; SEQ ID NO:3.

oNJ338; SEQ ID NO:4.

oNJ340; SEQ ID NO:5.

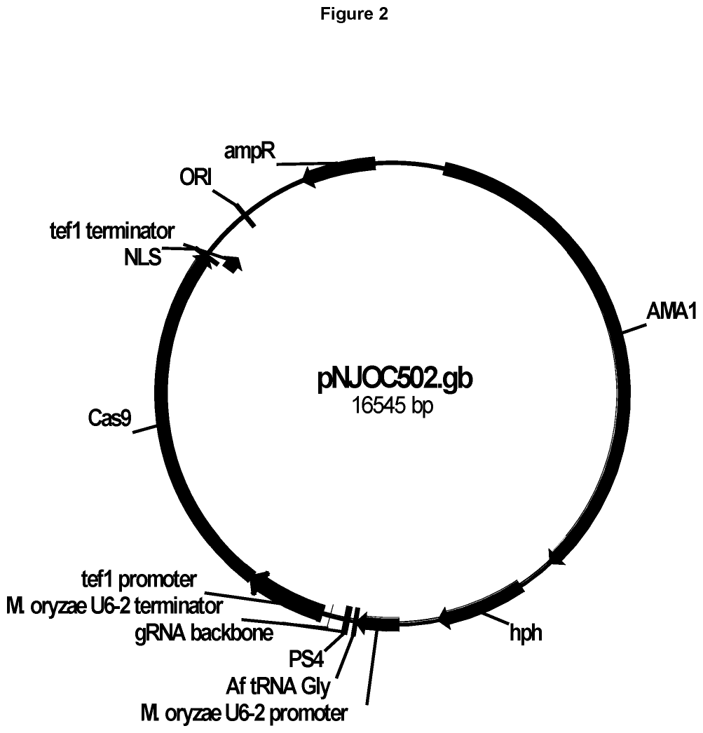

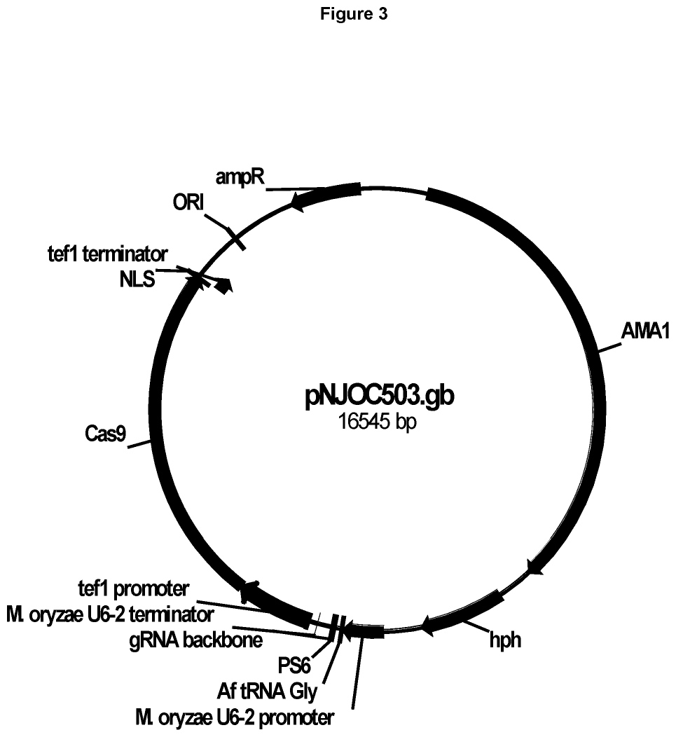

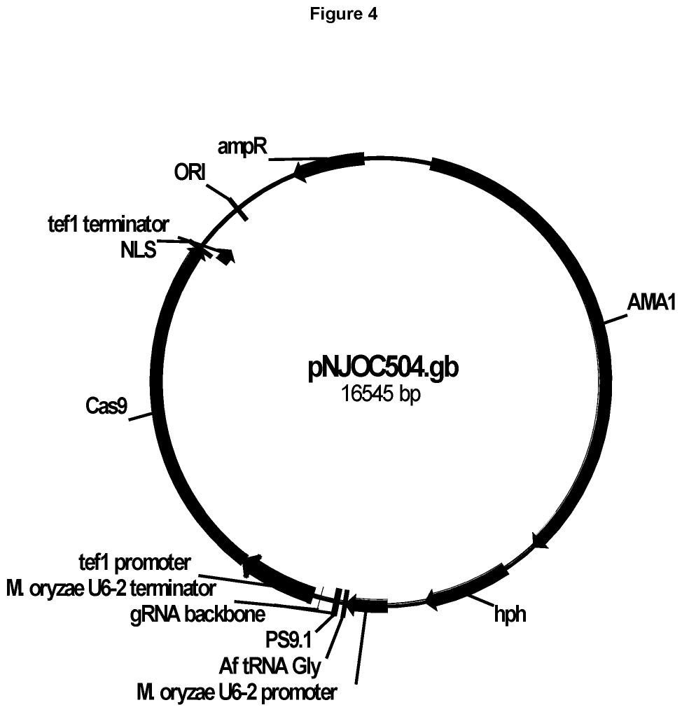

Assembly of protospacers. Protospacers were cloned into pSMAI290 using an NEBuilder® HiFi DNA Assembly Master Mix kit (New England Biolabs) in a total volume of 10 μl composed of 1× NEBuilder® HiFi Assembly Master Mix, 0.02 μmol of BgIII-digested pSMAI290, 0.5 μl of protospacer oligo (1 μM) and sterile Milli-Q H₂O to a final volume of 10 μl. The reactions were incubated at 50° C. for 60 minutes and then placed on ice. One μL of each reaction was used to transform 60 μL Stellar™ Competent Cells (Clontech Laboratories, Inc.) according to the manufacturer's instructions. Each transformation reaction was spread onto two 2×YT+Amp plates and incubated at 37° C. overnight. Putative transformant colonies were isolated from the selection plates and plasmid DNA was prepared from each one using a QIAprep Spin Miniprep kit (QIAGEN Inc.) and screened for insertion of the desired protospacer by sequencing using primer oNJ260 (SEQ ID NO:6). Plasmids having the correct protospacer sequence were labelled pNJ00502-504 (SEQ ID NOs:7-9, FIGS. 2-4) and saved for later use.

### Example 4: Co-Transformation of pNJ00502-504 and Single-Stranded Oligonucleotides

The purpose of this experiment was to examine if single-stranded oligonucleotides can be used as donor DNA for genome editing using a polynucleotide-guided nuclease, such as MAD7, Cas9 etc. The pNJ00502-pNJ00504 are autonomously replicating plasmids (contain AMA1) that express Cas9, an sgRNA construct that targets a specific sequence of the ACE3 locus in T. reesei and a hph selection marker (hygromycin B resistance). The oligonucleotides were designed such that the entire target sequence at the ACE3 locus would be replaced with a HindIII site (to facilitate screening by PCR and HindIII digestion) upon recombination between the donor DNA and the target locus. TrGMEr62-24a2-1 protoplasts were thawed on ice. For each transformation, approx. 2 μg of plasmid DNA and 5 μl single-stranded oligonucleotide (100 μM, synthesized by Thermo Fisher Scientific) were added to 100 μl of thawed protoplast solution and mixed gently. PEG buffer (250 μl) was added, and the reaction was mixed and incubated at 34° C. for 30 minutes. Following transformation, 1 ml of STC was added to each transformation reaction and the contents were spread onto PDA+1 M sucrose plates and incubated overnight at 30° C. The next day, an overlay consisting of PDA+hygromycin B was added to a final concentration of 10 μg/ml hygromycin B and the plates were incubated at 30° C. for 5-7 days. Approximately, 100-200 transformants were obtained for each transformation. To determine editing frequency, a handful of hygromycin-resistant colonies were picked from each transformation plate and transferred to PDA plates and incubated at 30° C. for 5-7 days. For each transformant, spores were collected with a sterile 1 μl inoculation loop and suspended in 20 μl of Dilution buffer (PHIRE™ Plant Direct PCR Kit, Thermo Scientific) in a thin-walled PCR tube. A region covering the target sites was amplified using the PHIRE™ Plant Direct PCR Kit (Thermo Scientific) with oNJ456 and oNJ459 as forward and reverse primers.

oNJ456; SEQ ID NO:10

oNJ459; SEQ ID NO:11

Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and sterile Milli-Q H₂O to a final volume of 20 μl. The reactions were incubated in a Bio-Rad C1000 Touch™ Thermal Cycler (Bio-Rad Laboratories) programmed for 1 cycle at 98° C. for 3 minutes; 40 cycles each at 98° C. for 5 seconds and 72° C. for 1 minute 20 seconds; and one cycle at 72° C. for 5 minutes.

To identify edited transformants, the PCR products were digested with HindIII. Edited transformants should give rise to two bands following PCR/HindIII digestion whereas non-edited transformants should only give rise to a single band (no HindIII site present). Each HindIII digestion reaction was composed of the following: 5 μl PCR product, 1× CutSmart buffer (New England Biolabs), 6 units of HindIII-HF enzyme (New England Biolabs) and sterile Milli-Q H₂O to a 20 μl final volume. The HindIII digestions were incubated at 37° C. for 1 hour and then analyzed by 1% agarose gel electrophoresis using TBE buffer. The results are shown in table 1. It was possible to obtain high editing efficiencies for all three protospacers/single-stranded oligonucleotide donor DNA combinations tested.

TABLE 1

| | | | | | Transformants |
|---|---|---|---|---|---|
| Transformation | Plasmid | Protospacer | Repair DNA | % edited | screened |
| 1 | pNJOC502 | PS4 | oNJ344 | 83 | 6 |
| | | (SEQ ID NO:12) | (SEQ ID NO:15) | | |
| 2 | pNJOC503 | PS6 | oNJ346 | 100 | 5 |
| | | (SEQ ID NO:13) | (SEQ ID NO:16) | | |
| 3 | pNJOC504 | PS9.1 | oNJ348 | 60 | 5 |
| | | (SEQ ID NO:14) | (SEQ ID NO:17) | | |

### Example 5: Delivery of SNVs Up to 43 bp from the Cut Site

Several studies have shown that there is a negative correlation between the distance from the cut site and incorporation of mutations using single-stranded oligonucleotides as donor DNA [(Inui, M., et al., Rapid generation of mouse models with defined point mutations by the CRISPR/Cas9 system. Sci Rep. 2014; 4:5396), (Wang, K., et al., Efficient Generation of Orthologous Point Mutations in Pigs via CRISPR-assisted ssODN-mediated Homology-directed Repair. Mol Ther Nucleic Acids. 2016 November; 5(11): e396), (Paquet, D., et al., Efficient introduction of specific homozygous and heterozygous mutations using CRISPR/Cas9. Nature, 2016. 533: p. 125-129)]. In pigs (porcine fetal fibroblasts), Wang et al. (2016) demonstrated that a mutation-to-cut distance of 11 bp resulted in a remarkable difference in homology-directed repair efficiency between two point mutations [Wang, K., et al., Efficient Generation of Orthologous Point Mutations in Pigs via CRISPR-assisted ssODN-mediated Homology-directed Repair. Mol Ther Nucleic Acids. 2016 November; 5(11): e396]. Likewise, for human induced pluripotent stem cells, Paquet et al. (2016) state that cut-to-mutation distance needs to be minimized for efficient homozygous mutation incorporation [Paquet, D., et al., Efficient introduction of specific homozygous and heterozygous mutations using CRISPR/Cas9. Nature, 2016. 533: p. 125-129]. Similarly, for mice, Inui et al. (2014) report that the distance between the modification site and gRNA target site was a significant parameter affecting the efficiency of the substitution [Inui, M., et al., Rapid generation of mouse models with defined point mutations by the CRISPR/Cas9 system. Sci Rep. 2014; 4:5396].

For directed mutagenesis or genome editing in Saccharomyces cerevisiae, Horwitz et al. (2015) report that the site targeted for cutting should as close as possible to the site of the desired mutation. Furthermore, to disrupt the Cas9p target site in the donor DNA and simultaneously improve the chances that recombination events include the desired mutation, Horwitz et al. (2015) made silent changes in the codons between the target site and the point mutation (a “heterology block”) [Horwitz, A. A., et al., Efficient Multiplexed Integration of Synergistic Alleles and Metabolic Pathways in Yeasts via CRISPR-Cas. Cell Syst. 2015. 1(1): p. 88-96].

The purpose of this experiment was to examine how the distance between the cut site and the intended mutation affects the frequency of mutation incorporation. The pNJ00503 CRISPR/Cas9 targeting plasmid was used and different single-stranded oligonucleotides (ordered as Ultramers® from IDT, Integrated DNA Technologies) were tested as donor DNA: oNJ499; SEQ ID NO:18.

oNJ500; SEQ ID NO:19.

oNJ501; SEQ ID NO:20.

oNJ502; SEQ ID NO:21.

oNJ503; SEQ ID NO:22.

oNJ504; SEQ ID NO:23.

oNJ505; SEQ ID NO:24.

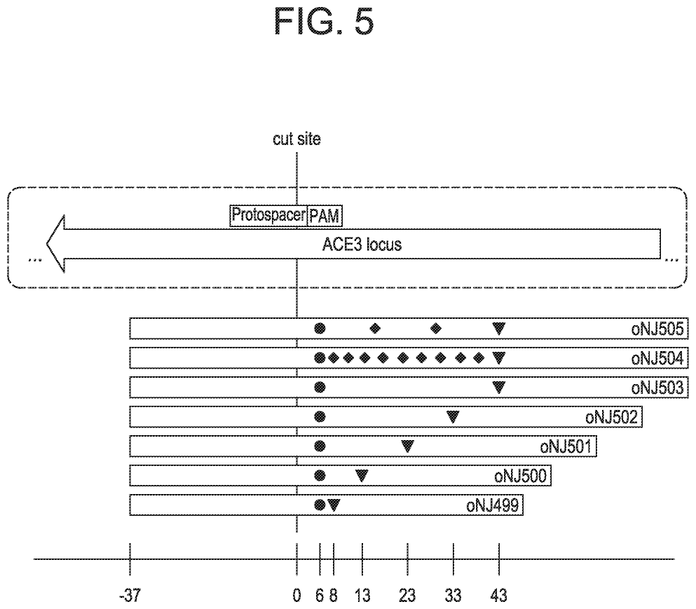

All oligonucleotides were designed to change the sequence of the NGG PAM site at the target locus to NGT to prevent Cas9 recognition and re-cutting in edited transformants. An additional mutation was incorporated into the oligos corresponding to insertion of the mutation 8 bp, 13 bp, 23 bp, 33 bp or 43 bp downstream from the Cas9 cut site (indicated by ▾ in FIG. 5). All oligonucleotides contained 42 unmodified nucleotides (nt) on the 5′ side of the PAM mutation and 40 unmodified nt on the 3′ side of the mutation being investigated for mutation incorporation (indicated by ▾ in FIG. 5). Furthermore, based upon the recommendations by Horwitz et al. (2015), two additional oligos containing “buffer mutations” (indicated by ▾ in FIG. 5) between the PAM mutation and the mutation 43 nt from the cut site were also tested to see if addition of extra “buffer mutations” can increase the rate of mutation incorporation further away from the cut site (FIG. 5).

TrGMEr62-24a2-1 protoplasts were thawed on ice. For each transformation, approx. 2 μg of pNJ00503 plasmid DNA and 5 μl single-stranded oligonucleotide (100 μM) were added to 100 μl of thawed protoplast solution and mixed gently. PEG buffer (250 μl) was added, and the reaction was mixed and incubated at 34° C. for 30 minutes. Following transformation, 1 ml of STC was added to each transformation reaction and the contents were spread onto PDA+1 M sucrose plates and incubated overnight at 30° C. The next day, an overlay consisting of PDA+hygromycin B was added to a final concentration of 10 μg/ml hygromycin B and the plates were incubated at 30° C. for 5-7 days. Approximately, 100-200 transformants were obtained for each transformation. To determine editing frequency, 6-12 hygromycin-resistant transformants were picked from each transformation plate and transferred to PDA plates and incubated at 30° C. for 5-7 days. For each transformant, spores were collected with a sterile 1 μl inoculation loop and suspended in 20 μl of Dilution buffer (PHIRE™ Plant Direct PCR Kit, Thermo Scientific) in a thin-walled PCR tube. A region covering the target site was amplified using the PHIRE™ Plant Direct PCR Kit (Thermo Scientific) with oNJ456 (SEQ ID NO:10) and oNJ459 (SEQ ID NO:11) as forward and reverse primers.

Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and sterile Milli-Q H₂O to a final volume of 20 μl. The reactions were incubated in a Bio-Rad C1000 Touch™ Thermal Cycler (Bio-Rad Laboratories) programmed for 1 cycle at 98° C. for 3 minutes;

40 cycles each at 98° C. for 5 seconds and 72° C. for 1 minute 20 seconds; and one cycle at 72° C. for 5 minutes. To identify edited transformants and to estimate the frequency of transfer of the desired mutations (relative to editing by homology-directed repair), the PCR products were sequenced using the forward primer oNJ474 (SEQ ID NO:25).

TABLE 2

| | Distance | | Number of | | |
|---|---|---|---|---|---|
| | between | | transformants | Number of | |
| | desired | | edited by | transformants | |
| | mutation | Additional | homology | edited by HDR and | Transfer |
| Oligo/SEQ | and cut site | “buffer | directed | all mutations | efficiency |
| ID NO: | (nucleotides, nt) | mutations” | repair (HDR) | incorporated | [%] |
| oNJ499/18 | 8 | — | 9 | 9 | 100 |
| oNJ500/19 | 13 | — | 12 | 12 | 100 |
| oNJ501/20 | 23 | — | 10 | 10 | 100 |
| oNJ502/21 | 33 | — | 10 | 6 | 60 |
| oNJ503/22 | 43 | — | 12 | 10 | 83 |
| oNJ504/23 | 43 | 2 | 11 | 10 | 91 |
| oNJ505/24 | 43 | 9 | 6 | 6 | 100 |

It was possible to transfer mutations far away from the cut site (up to 43 bp in this experiment) with high efficiencies (60-100% of the edited transformants contained all the intended mutations). The mutation transfer efficiencies were much higher than the efficiencies reported for mammalian cells using single-stranded oligonucleotides [(Inui, M., et al., Rapid generation of mouse models with defined point mutations by the CRISPR/Cas9 system. Sci Rep. 2014; 4:5396), (Wang, K., et al., Efficient Generation of Orthologous Point Mutations in Pigs via CRISPR-assisted ssODN-mediated Homology-directed Repair. Mol Ther Nucleic Acids. 2016 November; 5(11): e396), (Paquet, D., et al., Efficient introduction of specific homozygous and heterozygous mutations using CRISPR/Cas9. Nature, 2016. 533: p. 125-129)] or yeast using double-stranded oligonucleotides [Horwitz, A. A., et al., Efficient Multiplexed Integration of Synergistic Alleles and Metabolic Pathways in Yeasts via CRISPR-Cas. Cell Syst. 2015. 1(1): p. 88-96].

### Example 6: Delivery of SNVs Up to 63 bp Away from the Cut Site

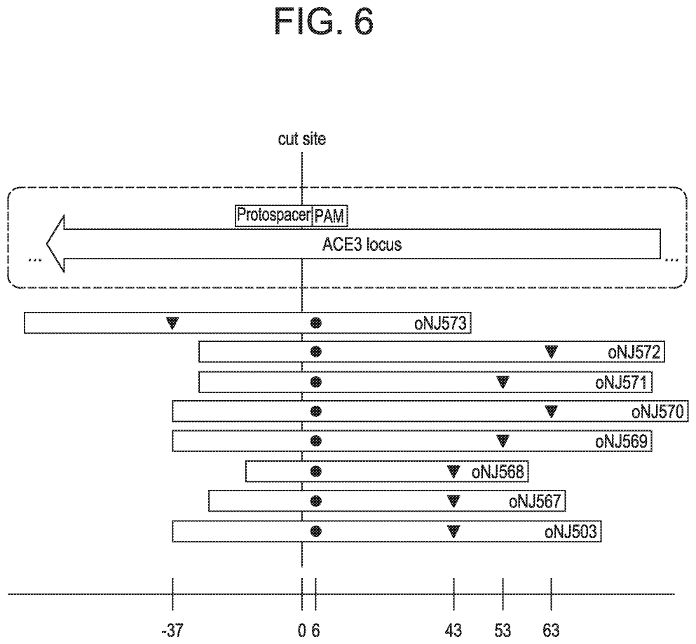

Based on the promising results mentioned in the example above, it was decided to test if mutations could be transferred even further away from the cut site and to what extent the amount of homology on each side of the mutations affect the transfer efficiency (FIG. 6). Eight different single-stranded oligonucleotides (ordered as Ultramers® from IDT, Integrated DNA Technologies) were tested in this experiment (all without “buffer mutations”). TrGMEr62-24a2-1 protoplasts were thawed on ice. For each transformation, approx. 2 μg of pNJ00503 plasmid DNA and 5 μl single-stranded oligonucleotide (100 μM) were added to 100 μl of thawed protoplast solution and mixed gently. PEG buffer (250 μl) was added, and the reaction was mixed and incubated at 34° C. for 30 minutes. Following transformation, 1 ml of STC was added to each transformation reaction and the contents were spread onto PDA+1 M sucrose plates and incubated overnight at 30° C. The next day, an overlay consisting of PDA+hygromycin B was added to a final concentration of 10 μg/ml hygromycin B and the plates were incubated at 30° C. for 5-7 days. Approximately, 100-200 transformants were obtained for each transformation. This time, Whatman™ 150 mm sterile filter papers (GE Healthcare UK Limited) were used to transfer mycelia from the PDA+1M sucrose+hygromycin B plates to PDA+1M sucrose plates. The plates were incubated at 30° C. for 5-7 days. To get an estimate of the overall editing and mutation transfer efficiencies, the spores from the plates from each transformation were collected in sterile Milli-Q H₂O and 1 μl spore suspension was added to 20 μl of Dilution buffer (PHIRE™ Plant Direct PCR Kit, Thermo Scientific) in a thin-walled PCR tube. A region covering the target site was amplified using the PHIRE™ Plant Direct PCR Kit (Thermo Scientific) with oNJ456 (SEQ ID NO:10) and oNJ459 (SEQ ID NO:11) as forward and reverse primers. Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and sterile Milli-Q H₂O to a final volume of 20 μl. The reactions were incubated in a Bio-Rad C1000 Touch™ Thermal Cycler (Bio-Rad Laboratories) programmed for 1 cycle at 98° C. for 3 minutes; 40 cycles each at 98° C. for 5 seconds and 72° C. for 1 minute 20 seconds; and one cycle at 72° C. for 5 minutes. To identify edited transformants and to estimate the frequency of transfer of the desired mutations, the PCR products were used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. For each sample, 100,000 trimmed reads were sampled using the Sample Reads module. Reads were mapped to a model of the ACE3 locus (SEQ ID NO:2) using the Map Reads to Reference module with a high-stringency setting. Overall, 85-96% of the reads were successfully mapped producing a 100% coverage of the model. Editing and transfer of mutations were analyzed with the Basic Variant Detection module and the results are summarized in the table below.

TABLE 3

| | Distance | Number of | | | |
|---|---|---|---|---|---|
| | between | contiguous, | | | |
| | desired | unmodified nt at | | % edited by | |
| | mutation | the ends of the | | HDR and all | Transfer |
| Oligo/SEQ | and cut site | oligonucleotide | % edited by | mutations | efficiency |
| ID NO: | (nucleotides, nt) | (5′/3′) | HDR | incorporated | [%] |
| oNJ503/22 | 43 | 42/40 | 93 | 78 | 84 |
| oNJ567/26 | 43 | 32/30 | 95 | 78 | 82 |
| oNJ568/27 | 43 | 22/20 | 80 | 54 | 68 |
| oNJ569/28 | 53 | 42/40 | 93 | 29 | 31 |
| oNJ570/29 | 63 | 42/40 | 93 | 68 | 73 |
| oNJ571/30 | 53 | 32/30 | 92 | 34 | 37 |
| oNJ572/31 | 63 | 32/30 | 91 | 36 | 39 |
| oNJ573/32 | 37 | 40/40 | 95 | 57 | 60 |
| | (upstream cut | | | | |
| | site) | | | | |

Using single-stranded oligonucleotides, it was possible to introduce mutations up to 63 bp from the cut site with high efficiencies as 31-84% of the edited transformants contained all the desired mutations (PAM mutation and the other mutation inserted at various distances from the cut site). Mutation transfer was efficient, even when the number of contiguous, unmodified nucleotides outside the intended mutations was reduced to ˜30 bp or ˜20 bp. Overall, the results from example 5 and example 6 demonstrated that mutations far away from the cut site can be introduced with surprisingly high efficiencies using single-stranded oligonucleotides. These are important results as it may not always be possible to find a good protospacer and a PAM sequence close to the target site of interest. For instance, for sequences containing 25% of each of the bases A, T, C and G, a four nucleotide TTTN or CTTN PAM sequence (where N is A, T, C or G) for an enzyme such as MAD7 (MAD7 shows preference for TTTV or CTTV PAM sites) would be expected to be present approx. every 64 bp for each strand of DNA or once every 32 bp overall. These results expand the amount of sequences, which can be efficiently targeted for CRISPR-mediated mutagenesis and genome editing using single-stranded oligonucleotides.

### Example 7: CRISPR/Cas9 and Single-Stranded Oligonucleotide Mediated Editing Using Reduced Amount of Single-Stranded Oligonucleotides

For high-throughput mutagenesis work using single-stranded oligonucleotides it would be advantageous if less oligonucleotide could be used per transformation as it would reduce the overall cost by allowing for re-use of the same oligo for multiple purposes and/or synthesis of the oligos at a smaller scale (lower price per nucleotide). Consequently, we wanted to test how the amount of single-stranded oligonucleotide affected transformation and editing efficiency. TrGMEr62-24a2-1 protoplasts were thawed on ice. For each transformation, approx. 2 μg of pNJ00503 plasmid DNA and 10-500 μmol oNJ346 (SEQ ID NO:16) single-stranded oligonucleotide (corresponding to 0.1-5 μl of a 100 μM stock) were added to 100 μl of thawed protoplast solution and mixed gently. PEG buffer (250 μl) was added, and the reaction was mixed and incubated at 34° C. for 30 minutes. Following transformation, 1 ml of STC was added to each transformation reaction and the contents were spread onto PDA+1 M sucrose plates and incubated overnight at 30° C. The next day, an overlay consisting of PDA+hygromycin B was added to a final concentration of 10 μg/ml hygromycin B and the plates were incubated at 30° C. for 5-7 days. Approximately, 100-200 transformants were obtained for each transformation, except for the transformation with 10 μmol of oNJ346 which only gave ˜50 transformants. Whatman™ 150 mm sterile filter papers (GE Healthcare UK Limited) were used to transfer mycelia from the PDA+1M sucrose+hygromycin B plates to PDA+1M sucrose plates. The plates were incubated at 30° C. for 5-7 days. The spores from the plates from each transformation were collected in sterile Milli-Q H₂O and 1 μl spore suspension was added to 20 μl of Dilution buffer (PHIRE™ Plant Direct PCR Kit, Thermo Scientific) in a thin-walled PCR tube. A region covering the target site was amplified using the PHIRE™ Plant Direct PCR Kit (Thermo Scientific) with oNJ456 and oNJ459 as forward and reverse primers. Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and sterile Milli-Q H₂O to a final volume of 20 μl. The reactions were incubated in a Bio-Rad C1000 Touch™ Thermal Cycler (Bio-Rad Laboratories) programmed for 1 cycle at 98° C. for 3 minutes; 40 cycles each at 98° C. for 5 seconds and 72° C. for 1 minute 20 seconds; and one cycle at 72° C. for 5 minutes. The PCR products were used to create paired-end sequencing libraries and sequenced using 2×150 bp chemistry on a NEXTSEQ™ 500 system (Illumina Inc.). Sequence analysis was performed with the CLC Genomics Workbench version 11.0.1 (QIAGEN). Reads were trimmed using the Trim Reads module. For each sample, 100,000 trimmed reads were sampled using the Sample Reads module. Reads were mapped to a model of the ACE3 locus (SEQ ID NO:2) using the Map Reads to Reference module with a high-stringency setting. Overall, 85-94% of the reads were successfully mapped producing a 100% coverage of the model. Editing (i.e. replacement of the target sequence with a HindIII site) was analyzed using the InDels and Structural Variants module. The results are shown in the table below:

TABLE 4

| Amount of oNJ346 | Total number of | Editing |
|---|---|---|
| used [pmol] | transformants | frequency [%] |
| 500 | ~200 | 93 |
| 200 | ~200 | 92 |
| 100 | ~200 | 95 |
| 50 | ~200 | 87 |
| 20 | ~100 | 98 |
| 10 | ~50 | 45 |

The amount of oNJ346 single-stranded oligonucleotide could be decreased to 20 μmol without negatively affecting editing efficiency (>87%) and still main a high transformation efficiency. This enables use of the same oligo for multiple transformations and allows for oligo synthesis at a smaller scale.

### Example 8: Aspergillus oryzae Protoplast Generation

Aspergillus oryzae transformation was performed according to Christensen et al., 1988, Biotechnology 6: 1419-1422. In short, A. oryzae mycelia were grown in a rich nutrient broth. The mycelia were separated from the broth by filtration. The enzyme preparation Glucanex® (Novozymes A/S) was added to the mycelia in an osmotically stabilizing buffer such as 1.2 M MgSO4 buffered to pH 5.0 with sodium phosphate. The suspension was incubated for 60 minutes at 37° C. with agitation. The protoplasts were filtered through Miracloth® (Calbiochem Inc.) to remove mycelial debris. The protoplasts were harvested and washed twice with STC. The protoplasts were then resuspended in a suitable volume of STC corresponding to approx. 10⁷ protoplasts/ml.

### Example 9: CRISPR/Mad7 Backbone Vector pAT3630

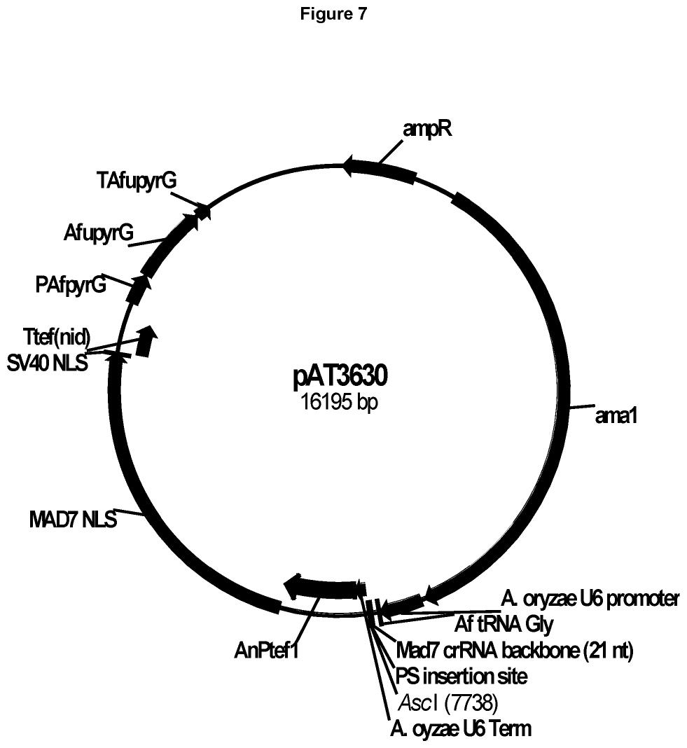

Plasmid pAT3630 (SEQ ID NO:33, FIG. 7) is a CRISPR/Mad7 expression plasmid used to clone in protospacers into Ascl digested pAT3630 using an NEBuilder® HiFi DNA Assembly Cloning Kit (New England Biolabs Inc.). Plasmid pAT3630 contains a Eubacterium rectale Mad7 protein coding sequence (nucleotides 8776-12564 in pAT3630) and codon-optimized for use in Aspergillus oryzae and a SV40 nuclear localization signal containing an extra proline residue and a DNA sequence for a stop codon (NLS; nucleotides 12565-12591) at the 3′ end of the E. rectale Mad7 open reading frame to ensure that Mad7 would be localized to the nucleus. The expression of the E. rectale Mad7 is under control of the Aspergillus nidulans tef1 promoter (nucleotides 7890-8775) and terminator (nucleotides 12592-12996) from pFC330-333 (Nødvig et al., 2015, PLoS One 10(7): 1-18). Plasmid pAT3630 also has all the elements for single guide RNA (sgRNA) expression, which consists of the Aspergillus oryzae U6 promoter (nucleotides 7116-7623), Aspergillus fumigatus tRNAgly(GCC)1-6 sequence with the region downstream the structural tRNA removed (nucleotides 7624-7714), E. rectale single guide RNA sequence (nucleotides 7715-7735), Ascl restriction enzyme recognition sequence (nucleotides 7736-7743), and Aspergillus oryzae U6 terminator (nucleotides 7744-7881). For selection in A. oryzae, plasmid pAT3630 contains the pyrG gene incl. promoter and terminator from Aspergillus fumigatus (nucleotides 13174-14612), conferring the ability to grow without addition of uridine, and the autonomous maintenance in Aspergillus (AMA1) sequence (Gems et al., 1991, Gene 98: 61-67) (nucleotides 1359-7079) for extrachromosomal replication of pAT3630 in A. oryzae. The single guide RNA and the Mad7-SV40 NLS expression elements in pAT3630 were confirmed by DNA sequencing on an Illumina MiSeq system.

### Example 10: Construction of pAT3720

Plasmid preparation. Plasmid pAT3630 was digested with the restriction enzyme Ascl (Ascl, New England Biolabs). The restriction reaction contained: 4 μg of pAT3630 plasmid DNA, 1× CutSmart® buffer, 10 units of Ascl, and sterile Milli-Q water up to 50 μl final volume. The reaction was incubated at 37° C. for 1 hour. The Ascl enzyme was then inactivated by heating to 80° C. for 20 minutes.

Protospacer design. A twenty-one base-pairs protospacer (SEQ ID:34) was designed for the wA locus (SEQ ID NO:35) to direct the Mad7 enzyme to the target site and create a double stranded break. The protospacer was selected by finding an appropriate protospacer adjacent motif (PAM) with the sequence TTTV, where V represents nucleotides A, C, or G. Once an appropriate PAM site was identified, the twenty-one base-pairs immediately adjacent to the 3′ side of the PAM site were selected as the protospacer. Protospacers that contained more than three contiguous T nucleotides were rejected to avoid possible stuttering of RNA polymerase.

The protospacer with its extension sequences used for cloning (oAT3858) was synthesized as a single-stranded oligonucleotide by Integrated DNA Technologies, Inc. The protospacer oligonucleotide was diluted to a final working concentration of 1 μM:

oAT3858 (SEQ ID NO: 36):
AATTTCTACTCTTGTAGATacgatggtgctgatggctactttttttt
ttgagcatttatcagcttg

Insertion of protospacer into pAT3630. The protospacer was cloned into pAT3630 using an NEBuilder® HiFi DNA Assembly Master Mix kit (New England Biolabs) in a total volume of 10 μl composed of 1× NEBuilder® HiFi Assembly Master Mix, 50 ng of Ascl-digested pAT3630, 0.5 μl of protospacer oligo (1 μM) and sterile Milli-Q H₂O to a final volume of 10 μl. The reaction was incubated at 50° C. for 60 minutes and then placed on ice. One μL of the reaction was used to transform 60 μL Stellar™ Competent Cells (Clontech Laboratories, Inc.) according to the manufacturer's instructions. The transformation reaction was spread onto two 2×YT+Amp plates and incubated at 37° C. overnight. Putative transformant colonies were isolated from the selection plates and plasmid DNA was prepared from each one using a QIAprep Spin Miniprep kit (QIAGEN Inc.) and screened for insertion of the desired protospacer by sequencing using primer oAT4025 (SEQ ID NO:37).

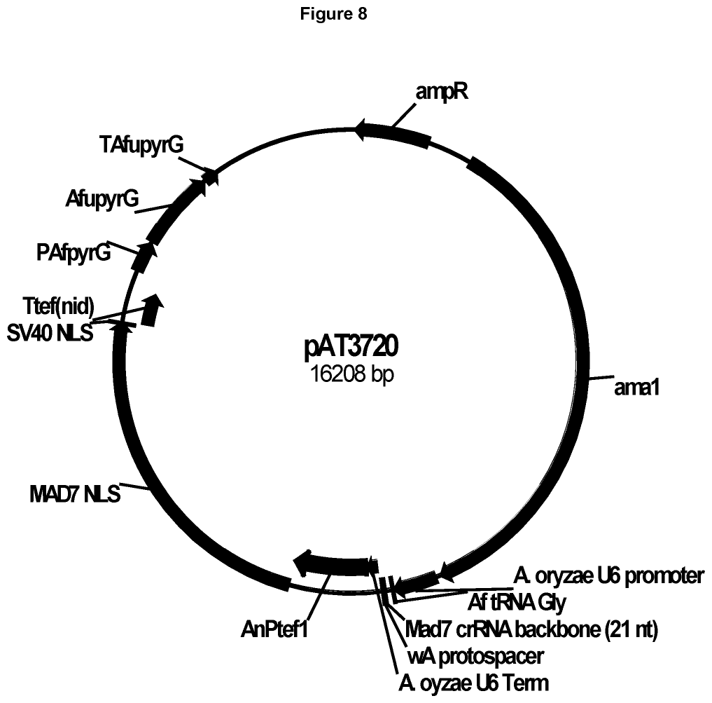

A plasmid having the correct protospacer sequence was labelled pAT3720 (SEQ ID NO:38, FIG. 8) and saved for later use.

### Example 11: Delivery of SNVs Up to 58 bp Away from the CRISPR/Mad7 Cut Site

Based on the promising results obtained using CRISPR/Cas9 mentioned in the examples above, it was decided to test if mutations could be transferred up to 58 bp away from the cut site using CRISPR/Mad7 to make the double strand break at the target site. The distance from the cut site reported in the table below is presented relative to position 19 3′ to the PAM site. Three different single-stranded oligonucleotides (oAT4070-oAT4072, ordered as Ultramers® from IDT, Integrated DNA Technologies, Inc.) were tested in this experiment. AT526 protoplasts were thawed on ice. For each transformation, approx. 100 ng of pAT3720 plasmid DNA and 5 μl single-stranded oligonucleotide (100 μM) were added to 100 μl of protoplast solution and mixed gently. PEG buffer (300 μl) was added, and the reaction was mixed and incubated at room temperature for 20 minutes. Following transformation, 6 ml of top agar solution (cooled to 40-50° C.) added to each transformation reaction and the contents were spread onto sucrose+urea plates and incubated overnight at 37° C. The next day, the plates were moved to 30° C. and incubated for 4-5 days and then left at room temperature for an additional 2-3 days. Approximately, 20-50 transformants were obtained for each transformation. For each transformation, spores from eight transformants were streaked onto sucrose+urea+triton plates and incubated at 37° C. for 4-6 days followed by incubation at room temperature for an additional 2-3 days. Spores from the sucrose+urea+triton plates were collected in sterile Milli-Q H₂O and 1 μl spore suspension was added to 30 μl of Dilution buffer (PHIRE™ Plant Direct PCR Kit, Thermo Scientific) in a thin-walled PCR tube. A region covering the target site was amplified using the PHIRE™ Plant Direct PCR Kit (Thermo Scientific) with oAT4074 (SEQ ID NO:39) and oAT4075 (SEQ ID NO:40) as forward and reverse primers.

Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and sterile Milli-Q H₂O to a final volume of 20 μl. The reactions were incubated in a Bio-Rad C1000 Touch™ Thermal Cycler (Bio-Rad Laboratories) programmed for 1 cycle at 98° C. for 3 minutes; 40 cycles each at 98° C. for 5 seconds and 72° C. for 1 minute 20 seconds; and one cycle at 72° C. for 5 minutes. To identify edited transformants and to estimate the frequency of transfer of the desired mutations (relative to editing by homology-directed repair), the PCR products were sequenced using the primer oAT4076 (SEQ ID NO:41).

TABLE 5

| | Distance | | | | |
|---|---|---|---|---|---|
| | between | | | | |
| | desired | Number of | Number of | Number of | |
| | mutation | contiguous, | transformants | transformants | |
| | and the | unmodified nt at | edited by | edited by | |
| | upstream | the ends of the | homology | HDR and all | Transfer |
| Oligo/SEQ | cut site | oligonucleotide | directed | mutations | efficiency |
| ID NO: | (nucleotides, nt) | (5′/3′) | repair (HDR) | incorporated | [%] |
| oAT4070/42 | 18 | 40/40 | 8 | 6 | 75 |
| oAT4071/43 | 38 | 40/40 | 8 | 5 | 63 |
| oAT4072/44 | 58 | 40/40 | 8 | 3 | 38 |

The transfer efficiencies obtained in this experiment with a sample size of 8 were comparable to the efficiencies observed in T. reesei using CRISPR/Cas9 and single-stranded oligonucleotides.

### Example 12: Transfer of SNVs Using CRISPR/Cas9 and Single-Stranded Oligonucleotides in Aspergillus niger

Based on the promising results obtained for CRISPR/Cas9 and CRISPR/Mad7, the delivery of SNVs using single-stranded oligonucleotides combined with CRISPR was tested in a ku70 disrupted Aspergillus niger host using Cas9 as the CRISPR nuclease. A total of 11 different genes were targeted for editing using CRISPR/Cas9 and single-stranded oligonucleotides as the donor DNA. Overall, SNV transfer efficiencies of 60-100% were obtained when transferring mutations 7-26 bp from the Cas9 cut site (data not shown). The results demonstrate that insertion of SNVs using single-stranded oligonucleotides combined with CRISPR Cas9 is also possible in A. niger.

### Example 13: CRISPR/Mad7 Backbone Vector pGMEr263

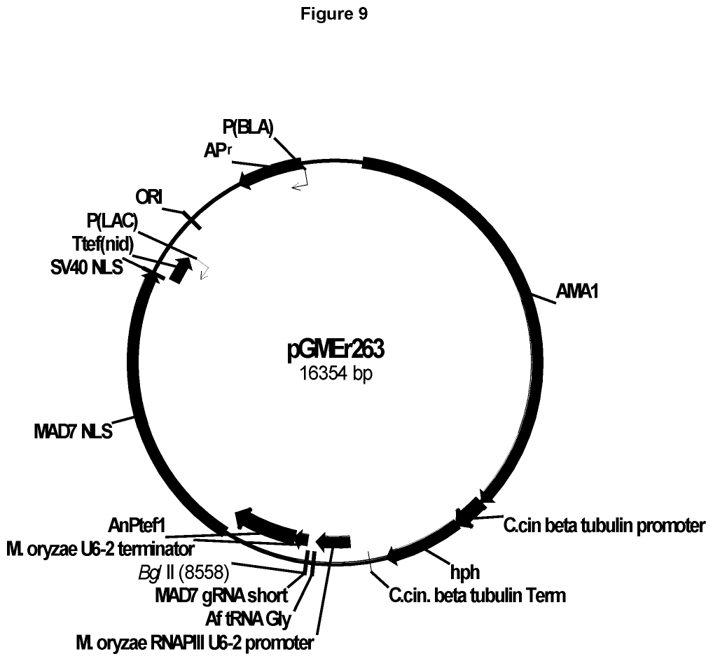

Plasmid pGMEr263 (SEQ ID NO:45, FIG. 9) is a CRISPR/MAD7 expression plasmid used to clone in protospacers into BgIII digested pGMEr263 using an NEBuilder® HiFi DNA Assembly Cloning Kit (New England Biolabs Inc.). Plasmid pGMEr263 contains a Eubacterium rectale Mad7 protein coding sequence (nucleotides 9663-13451 in pGMEr263) and codon-optimized for use in Aspergillus oryzae and a SV40 nuclear localization signal including an extra proline residue and a DNA sequence for a stop codon (nucleotides 13452-13478) at the 3′ end of the E. rectale Mad7 open reading frame to ensure that Mad7 would be localized to the nucleus.

The expression of the E. rectale Mad7 is under control of the Aspergillus nidulans tef1 promoter (nucleotides 8777-9662) and terminator (nucleotides 13,479-13,883) from pFC330-333 (Nodvig et al., 2015, PLoS One 10(7): 1-18). Plasmid pGMEr263 also has all the elements for single guide RNA (sgRNA) expression, which consists of the Magnaporthe oryzae U6-2 promoter (nucleotides 7949-8448), Aspergillus fumigatus tRNAgly(GCC)1-6 sequence with the region downstream the structural tRNA removed (nucleotides 8449-8539), E. rectale single guide RNA sequence (nucleotides 8540-8560), BgIII restriction enzyme recognition sequence (nucleotides 8557-8562), and M. oryzae U6-2 terminator (nucleotides 8562-8776). For selection in T. reesei, plasmid pGMEr263 contains the hygromycin phosphotransferase gene from pHT1 (Cummings et al., 1999, Curr. Genet. 36: 371) (nucleotides 6475-7506), conferring resistance to hygromycin B, and the autonomous maintenance in Aspergillus (AMA1) sequence (Gems et al., 1991, Gene 98: 61-67) (nucleotides 332-6056) for extrachromosomal replication of pGMEr263 in T. reesei. The single guide RNA and the Mad7-SV40 NLS expression elements in pGMEr263 were confirmed by DNA sequencing with a Model 377 XL Automated DNA Sequencer using dye-terminator chemistry.

### Example 14: Construction of pGMEr263proto1-Proto5

Plasmid vector preparation. Plasmid pGMEr263 was digested with the restriction enzyme BgIII (Anza™ 19 BgIII, Thermo Fisher Scientific). The restriction reaction contained: 15 μg of pGMEr263 plasmid DNA, 1× Anza™ buffer, 100 units of BgIII, and sterile Milli-Q water up to 200 μl final volume. The reaction was incubated at 37° C. for 3 hours. Following restriction enzyme digestion, the digest was subjected to 0.8% agarose gel electrophoresis in TBE buffer and the band representing the digested pGMEr263 was excised from the gel and purified using a NucleoSpin® Gel and PCR Clean-up kit (Macherey-Nagel) according to the manufacturer's instructions.

Protospacer design. Five different twenty-one base-pairs protospacers were designed for the ACE3 locus (SEQ ID NO:2) to direct the Mad7 enzyme to the target site and create a double stranded break. Protospacers were selected by finding an appropriate protospacer adjacent motif (PAM) with the sequence TTTV, where V represents nucleotides A, C, or G. Once an appropriate PAM site was identified, the twenty-one base-pairs immediately adjacent to the 3′ side of the PAM site were selected as the protospacer. Protospacers that contained more than three contiguous T nucleotides were rejected to avoid possible stuttering of RNA polymerase.

Each protospacer with its extension sequences used for cloning (U.S. Pat. Nos. 1,228,713, 1,228,715, 1,228,717, 1,228,719 and 1,228,721) was synthesized as a single-stranded oligonucleotide by Thermo Fisher Scientific, Inc. The underlined sequence in each oligonucleotide highlights the five twenty-one nucleotide protospacers. All protospacer oligonucleotides were diluted to a final working concentration of 1 μM:

1228713; SEQ ID NO:46

1228715; SEQ ID NO:47

1228717; SEQ ID NO:48

1228719; SEQ ID NO:49

1228721; SEQ ID NO:50

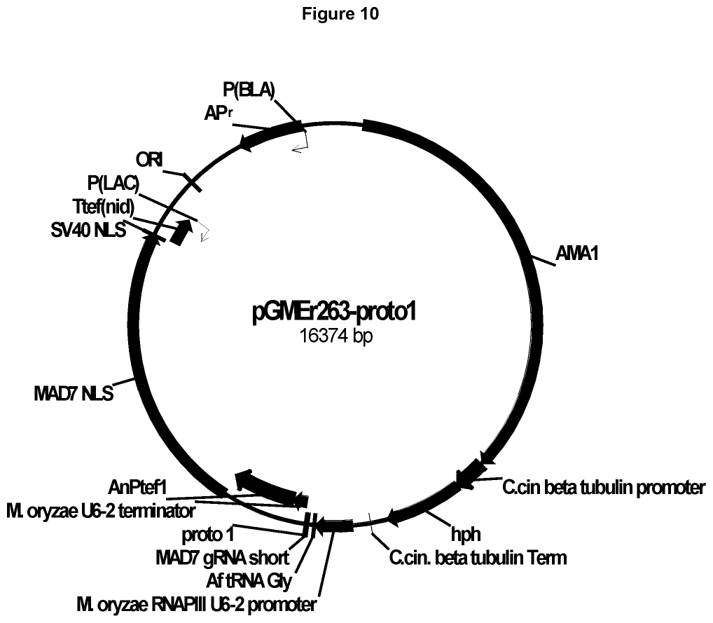

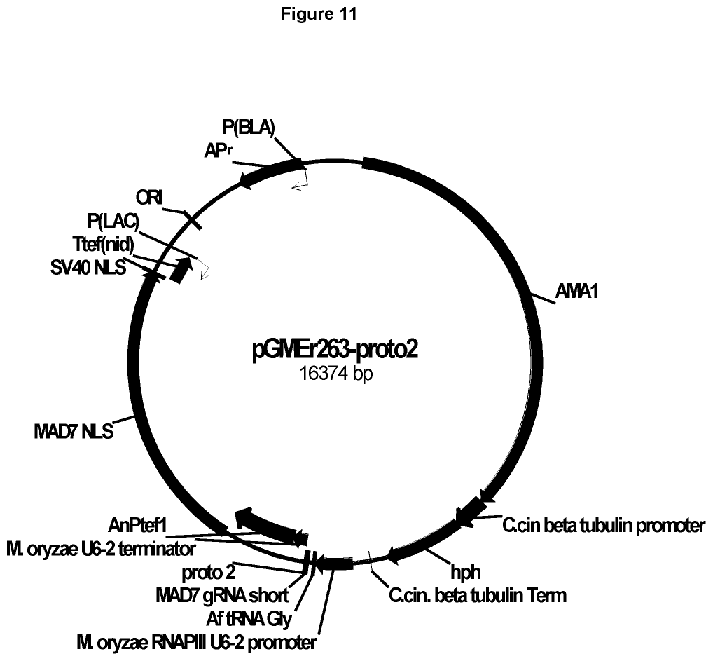

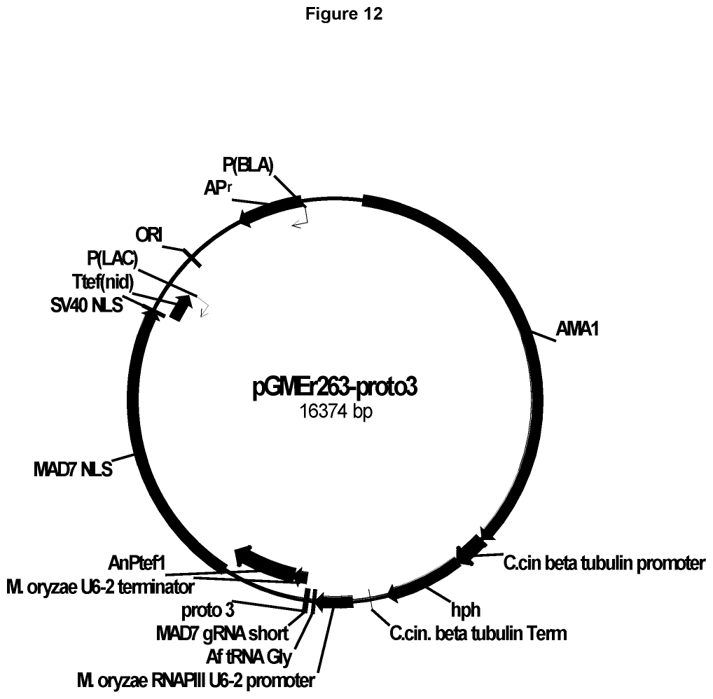

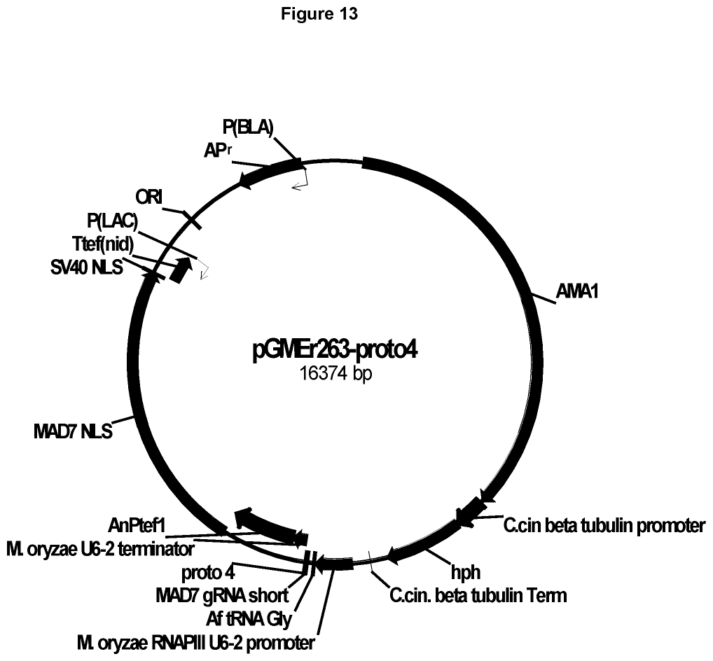

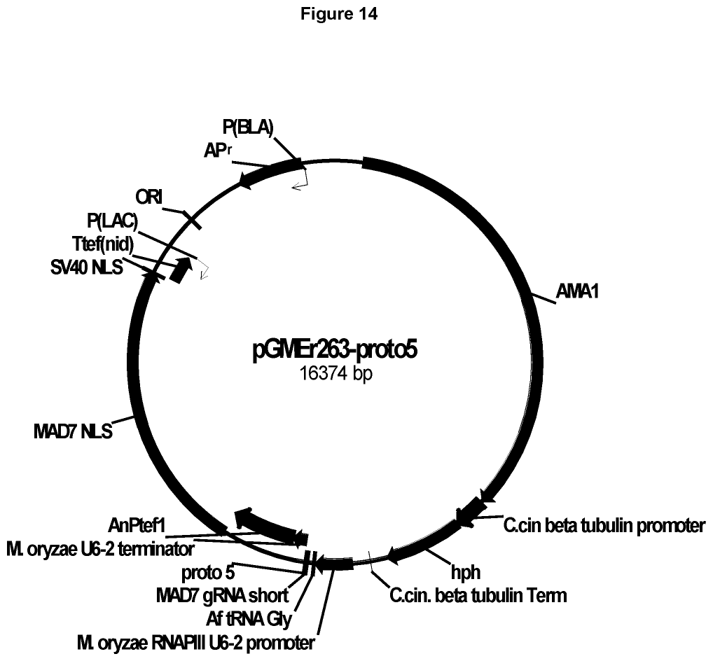

Assembly of protospacers. Protospacers were cloned into pGMEr263 using an NEBuilder® HiFi DNA Assembly Master Mix kit (New England Biolabs) in a total volume of 10 μl composed of 1× NEBuilder® HiFi Assembly Master Mix, 0.05 μmol of BgIII-digested pSMAI290, 1.0 μl of protospacer oligo (1 μM) and sterile Milli-Q H₂O to a final volume of 20 μl. The reactions were incubated at 50° C. for 15 minutes and then placed on ice. Two μL of each reaction was used to transform 50 μL Stellar™ Competent Cells (Clontech Laboratories, Inc.) according to the manufacturer's instructions. Each transformation reaction was spread onto two 2×YT+Amp plates and incubated at 37° C. overnight. Putative transformant colonies were isolated from the selection plates and plasmid DNA was prepared from each one using a QIAprep Spin Miniprep kit (QIAGEN Inc.) and screened for insertion of the desired protospacer by sequencing using primer 1228659 (SEQ ID NO:51). Plasmids having the correct protospacer sequence were labelled pGMEr263proto1-proto5 (SEQ ID NOs:52-56, FIGS. 10-14) and saved for later use.

### Example 15: Co-Transformation of pGMEr263proto1-Proto5 and Single-Stranded Oligonucleotides in T: reesei

The purpose of this experiment was to examine if single-stranded oligonucleotides can be used as donor DNA for genome editing using a polynucleotide-guided nuclease, such as MAD7, Cas9 etc. The pGMEr263proto1-pGMEr2630proto5 plasmids are autonomously replicating plasmids (contain AMA1) that express Mad7, an sgRNA construct that targets a specific sequence of the ACE3 locus in T. reesei and a hph selection marker (hygromycin B resistance). The oligonucleotides were designed such that the entire target sequence at the ACE3 locus would be replaced with a HindIII site (to facilitate screening by PCR and HindIII digestion) upon recombination between the donor DNA and the target locus. TrGMEr62-24a2-1 protoplasts were thawed on ice. For each transformation, approx. 2 μg of plasmid DNA and 3 μl single-stranded oligonucleotide (50 μM, synthesized by Thermo Fisher Scientific) were added to 100 μl of thawed protoplast solution and mixed gently. PEG buffer (250 μl) was added, and the reaction was mixed and incubated at 34° C. for 30 minutes. Following transformation, 1 ml of STC was added to each transformation reaction and the contents were spread onto PDA+1 M sucrose plates and incubated overnight at 34° C. The next day, an overlay consisting of PDA+hygromycin B was added to a final concentration of 10 μg/ml hygromycin B and the plates were incubated at 30° C. for 5-7 days. Approximately, 15-20 transformants were obtained for each transformation. To determine editing frequency, a handful of hygromycin-resistant colonies were picked from each transformation plate and transferred to PDA plates and incubated at 30° C. for 5-7 days. For each transformant, spores were collected with a sterile 1 μl inoculation loop and suspended in 20 μl of Dilution buffer (PHIRE™ Plant Direct PCR Kit, Thermo Scientific) in a thin-walled PCR tube. A region covering the target sites was amplified using the PHIRE™ Plant Direct PCR Kit (Thermo Scientific) with U.S. Pat. Nos. 1,228,586 and 1,228,587 as forward and reverse primers.

1228586 (SEQ ID NO:57)

1228587 (SEQ ID NO:58)

Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and sterile Milli-Q H₂O to a final volume of 20 μl. The reactions were incubated in a Bio-Rad C1000 Touch™ Thermal Cycler (Bio-Rad Laboratories) programmed for 1 cycle at 98° C. for 3 minutes; 40 cycles each at 98° C. for 5 seconds and 72° C. for 1 minute 20 seconds; and one cycle at 72° C. for 5 minutes.

To identify edited transformants, the PCR products were digested with HindIII. Edited transformants should give rise to two bands following PCR/HindIII digestion whereas non-edited transformants should only give rise to a single band (no HindIII site present). Each HindIII digestion reaction was composed of the following: 5 μl PCR product, 1× CutSmart buffer (New England Biolabs), 6 units of HindIII-HF enzyme (New England Biolabs) and sterile Milli-Q H₂O to a 20 μl final volume. The HindIII digestions were incubated at 37° C. for 1 hour and then analyzed by 1% agarose gel electrophoresis using TBE buffer. The results are shown in table 1. It was possible to obtain high editing efficiencies only for protospacer 1 (PS1; SEQ ID NO:60); all five protospacers/single-stranded oligonucleotide donor DNA combinations tested are listed in the table below:

TABLE 6

| | | | | | Transformants |
|---|---|---|---|---|---|
| Transformation | Plasmid | Protospacer | Repair DNA | % edited | screened |
| 1 | pGMEr263proto1 | PS1 | 1228583 | 75 | 12 |
| | | (SEQ ID NO:59) | (SEQ ID NO:64) | | |
| 2 | pGMEr263proto2 | PS2 | 1228584 | 18 | 11 |
| | | (SEQ ID NO:60) | (SEQ ID NO:65) | | |
| 3 | pGMEr263proto3 | PS3 | 1228585 | 50 | 2 |
| | | (SEQ ID N0:61) | (SEQ ID NO:66) | | |
| 4 | pGMEr263proto4 | PS4 | 1228582 | 0 | 12 |
| | | (SEQ ID NO:62) | (SEQ ID NO:67) | | |
| 5 | pGMEr263proto5 | PS5 | 1228581 | 42 | 12 |
| | | (SEQ ID NO:63) | (SEQ ID NO:68) | | |

### Example 16: Delivery of SNVs Via CRISPR/Mad7 Editing Up to 47 bp from the Cut Site Using Single-Stranded Oligonucleotides in T. reesei

The purpose of this experiment was to examine how the distance between the cut site and the intended mutation affects the frequency of mutation incorporation via CRISPR/Mad7 genome editing in Trichoderma reesei. The pGMEr263-proto1 CRISPR/Mad7 targeting plasmid was used and different single-stranded oligonucleotides (ordered as Ultramers® from IDT, Integrated DNA Technologies) were tested as donor DNA:

Oligo10 (SEQ ID NO:69)

Oligo11 (SEQ ID NO:70)

Oligo12 (SEQ ID NO:71)

Oligo13 (SEQ ID NO:72)

Oligonucleotides 10-13 were designed to change one nucleotide in the sequence of the TTTV PAM site at the target locus to TTGV and one nucleotide change in the protospacer 1 region to prevent Mad7 recognition and re-cutting in edited transformants. An additional mutation was incorporated into all the oligos corresponding to insertion of the desired SNV 20 bp, 32 bp, or 47 bp downstream, or −44 bp upstream from the Mad7 cut site. The distance from the cut site reported in the table below is presented relative to position 19 3′ to the PAM site. The table below show all the changed positions in each oligonucleotide used.

TABLE 7

| | | Protospacer | | | |
|---|---|---|---|---|---|
| | | mutations (position | | | |
| | PAM | number in | SNV distance from cut | 5′ flank | 3′flank |
| oligonucleotide | mutation | protospacer | site (nt) | (nt) | (nt) |
| Oligo10 | TTGG | +5 | 20 | 43 | 38 |
| Oligo11 | TTGG | +5 | 32 | 43 | 39 |
| Oligo12 | TTGG | +5 | 47 | 43 | 39 |
| Oligo13 | TTGG | +2 | −44 | 40 | 41 |

All oligonucleotides contained 61-39 unmodified nucleotides (nt) on the 5′ side and 33-41 unmodified nt on the 3′ side of the mutation (SNV) being investigated for mutation incorporation.

TrGMEr62-24a2-1 protoplasts were thawed on ice. For each transformation, approx. 2 μg of pGMER263-proto1 plasmid DNA and 3 μl single-stranded oligonucleotide (50 μM) were added to 100 μl of thawed protoplast solution and mixed gently. PEG buffer (250 μl) was added, and the reaction was mixed and incubated at 34° C. for 30 minutes. Following transformation, 1 ml of STC was added to each transformation reaction and the contents were spread onto PDA+1 M sucrose plates and incubated overnight at 34° C. overnight. The next day, an overlay consisting of PDA+hygromycin B was added to a final concentration of 10 μg/ml hygromycin B and the plates were incubated at 30° C. for 5-7 days. A number of transformants between 0 and 30 were obtained for each transformation. To determine editing frequency, a maximum of 15 hygromycin-resistant transformants were picked from each transformation plate and transferred to PDA plates and incubated at 30° C. for 5-7 days. For each transformant, spores were collected with a sterile 1 μl inoculation loop and suspended in 20 μl of Dilution buffer (PHIRE™ Plant Direct PCR Kit, Thermo Scientific) in a thin-walled PCR tube. A region covering the target site was amplified using the PHIRE™ Plant Direct PCR Kit (Thermo Scientific) with 1228586 (SEQ ID NO:57) and 1228587 (SEQ ID NO:58) as forward and reverse primers.

Each PCR reaction was composed of 1 μl of spore suspension, 10 μmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and sterile Milli-Q H₂O to a final volume of 20 μl. The reactions were incubated in a Bio-Rad C1000 Touch™ Thermal Cycler (Bio-Rad Laboratories) programmed for 1 cycle at 98° C. for 3 minutes; 40 cycles each at 98° C. for 5 seconds and 72° C. for 1 minute 15 seconds; and one cycle at 72° C. for 5 minutes. To identify edited transformants and to estimate the frequency of transfer of the desired mutations (relative to editing by homology-directed repair), the PCR products were sequenced using the forward primer oNJ459 (SEQ ID NO:11).

Table 8. Showing Oligos vs. Distance between desired mutation and cut site (number of nucleotides; Nt) (col 2); PAM mutations (col 3); Protospacer mutations (position number in protospacer) (col 4); Number of transformants edited by homology directed repair (HDR) (col 5); Number of transformants edited by HDR and all mutations incorporated (col 6).

| | | | | | Transfor- | |
|---|---|---|---|---|---|---|
| | | | | | mants | |
| | | | | | edited | |
| Oligo/ | | | | | and | Transfer |
| SEQ ID | | | | Transfor- | incorpo- | efficiency |
| NO: | Nt | PAM | Pos. | mants | rated | [%] |
| Oligo10/ | 20 | TTGG | +5 | 4 | 4 | 100 |
| Oligo11/ | 32 | TTGG | +5 | 3 | 3 | 100 |
| Oligo12/ | 47 | TTGG | +5 | 3 | 3 | 100 |
| Oligo13/ | −44 | TTGG | +2 | 5 | 5 | 100 |

It was possible to transfer mutations up to 47nt downstream and 44nt upstream of the Mad7 cut site with editing efficiencies between 36-100%. The mutation transfer efficiencies were much higher than the efficiencies reported for mammalian cells using single-stranded oligonucleotides [(Inui, M., et al., Rapid generation of mouse models with defined point mutations by the CRISPR/Cas9 system. Sci Rep. 2014; 4:5396), (Wang, K., et al., Efficient Generation of Orthologous Point Mutations in Pigs via CRISPR-assisted ssODN-mediated Homology-directed Repair. Mol Ther Nucleic Acids. 2016 November; 5(11): e396), (Paquet, D., et al., Efficient introduction of specific homozygous and heterozygous mutations using CRISPR/Cas9. Nature, 2016. 533: p. 125-129)] or yeast using double-stranded oligonucleotides [Horwitz, A. A., et al., Efficient Multiplexed Integration of Synergistic Alleles and Metabolic Pathways in Yeasts via CRISPR-Cas. Cell Syst. 2015. 1(1): p. 88-96].

### Example 17: Delivery of SNVs Up to 553 bp from the Cut Site Using CRISPR/Mad7 and dsDNA Fragments in T. reesei

The purpose of this experiment was to examine whether SNVs can be targeted with greater distance between the CRISPR cut site and the intended mutation. Seven different CRISPR/Mad7 targeting plasmids were tested individually together with different double-stranded DNA (ordered as Strings® from Geneart, or gene fragments from Twist Bioscience) as donor DNA.

All donor DNA were designed to change the sequence of the TTTV PAM site at the target locus as well as introduce a silent mutation in the protospacer to prevent Mad7 recognition and re-cutting in edited transformants. Mutations resulting in amino acid change were incorporated into the repair fragments. Furthermore, based upon the recommendations by Horwitz et al. (2015), additional “buffer mutations” between the PAM mutation and the desired mutation up to 553 bp from the cut site were included to see if addition of extra “buffer mutations” can increase the rate of mutation incorporation further away from the cut site.

TrGMEr62-24a2-1 protoplasts were thawed on ice. For each transformation, approx. 1.5 μg of CRISPR/Mad7 targeting plasmid DNA and 2-5 μg double-stranded donor DNA were added to 100 μl of thawed protoplast solution and mixed gently. PEG buffer (250 μl) was added, and the reaction was mixed and incubated at 37° C. for 30 minutes. Following transformation, 1 ml of STC was added to each transformation reaction and the contents were spread onto PDA+1 M sucrose plates and incubated overnight at 34° C. The next day, an overlay consisting of PDA+hygromycin B was added to a final concentration of 10 μg/ml hygromycin B and the plates were incubated at 30° C. for 5-7 days. Approximately, 2-8 transformants were obtained for each transformation. To determine editing frequency, hygromycin-resistant transformants were picked from each transformation plate and transferred to PDA plates and incubated at 30° C. for 5-7 days. For each transformant, spores were collected with a sterile 1 μl inoculation loop and suspended in 20 μl of Dilution buffer (PHIRE™ Plant Direct PCR Kit, Thermo Scientific) in a thin-walled PCR tube. A region covering the target site using forward and reverse primers just outside the region of the donor DNA homology was amplified using the PHIRE™ Plant Direct PCR Kit (Thermo Scientific).

Each PCR reaction was composed of 0.5 μl of spore suspension, 10 μmol of each primer, 5 μl of 2× PHIRE™ Plant PCR Buffer (PHIRE™ Plant Direct PCR Kit, Thermo Scientific), 0.2 μl of PHIRE™ Hot Start II DNA Polymerase (PHIRE™ Plant Direct PCR Kit, Thermo Scientific) and sterile Milli-Q H₂O to a final volume of 10 μl. The reactions were incubated in an Eppendorf Masterycler® Thermal Cycler (Eppendorf AG) programmed for 1 cycle at 98° C. for 5 minutes; 40 cycles each at 98° C. for 5 seconds, 67° C. for 5 seconds and 72° C. for 1 minute 20 seconds; and one cycle at 72° C. for 2 minutes. To identify edited transformants and to estimate the frequency of transfer of the desired mutations (relative to editing by homology-directed repair), the PCR products were sequenced using the forward primer and reverse primers inside the 5′ and 3′ flanks of the PCR product.

TABLE 9

| | Distance | | Number of | | |
|---|---|---|---|---|---|
| | between | | transformants | Number of | |
| | desired | | edited by | transformants | |
| Donor | mutation | Additional | homology | edited by HDR and | Transfer |
| ID/Proto | and cut site | “buffer | directed | mutations | efficiency |
| NO: | (nucleotides, nt) | mutations” | repair (HDR) | incorporated | [%] |
| 2K91Q/1 | 235 | 6 | 4 | 4 | 100 |
| 4A323T/2 | 546 | 4 | 3 | 1 | 33 |
| 4A323T/3 | 265 | 4 | 7 | 4 | 57 |
| 5T533I/4 | 222 | 6 | 8 | 7 | 88 |
| 5T533I/5 | 553 | 6 | 4 | 3 | 75 |
| 6R509P/6 | 190 | 4 | 7 | 7 | 100 |
| 6R509P/7 | 214 | 4 | 2 | 2 | 100 |

It was possible to transfer mutations quite far away from the cut site (up to 553 bp in this experiment) with high efficiencies (33-100% of the edited transformants contained all the intended mutations; Table 9). The mutation transfer efficiencies were high considering distance from the CRISPR/Mad7 cut site

1-14. (canceled)

15: A method for introducing one or more desired nucleotide modification(s) in at least one target sequence in the genome of a microorganism cell using a polynucleotide-guided endonuclease, said method comprising the steps of:
a) providing a microorganism host cell comprising at least one genome target sequence to be modified located in the vicinity of a protospacer adjacent motif (PAM) sequence for the polynucleotide-guided endonuclease;
b) transforming the microorganism host cell with:
i) the polynucleotide-guided endonuclease and at least one suitable guide polynucleotide for the at least one target sequence to be modified, OR one or more polynucleotide encoding the polynucleotide-guided endonuclease and encoding at least one suitable guide polynucleotide for the at least one target sequence to be modified, and
ii) at least one single-stranded oligonucleotide capable of hybridizing with the at least one genome target sequence, said oligonucleotide comprising the one or more desired nucleotide modification(s);
wherein the at least one genome target sequence to be modified is located from 20 to 250 nucleotides away from the protospacer adjacent motif (PAM) sequence for the polynucleotide-guided endonuclease in the genome of the microorganism host cell;
wherein the polynucleotide-guided endonuclease interacts with the guide polynucleotide and with a protospacer-complementary sequence in the genome and cuts or nicks the genome,
and wherein the at least one single-stranded oligonucleotide directs DNA repair across the cut or nick, thereby introducing the one or more desired modification(s) into the target sequence of the genome with an efficiency of at least:
70%, preferably at least 80% when the cut or nick is located 10-20 nucleotides from the desired nucleotide modification(s),
60% when the cut or nick is located 21-30 nucleotides from the desired nucleotide modification(s),
50% when the cut or nick is located 31-43 nucleotides from the desired nucleotide modification(s),
40% when the cut or nick is located 44-52 nucleotides from the desired nucleotide modification(s), or
30% when the cut or nick is located at least 53 nucleotides from the desired nucleotide modification(s).

16: The method of claim 15, wherein the microorganism host cell is a prokaryote or a eukaryote.

17: The method of claim 16, wherein the microorganism host cell is a prokaryote.

18: The method of claim 17, wherein the microorganism host cell is selected from the species of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis.

19: The method of claim 16, wherein the microorganism host cell is a filamentous fungal cell.

20: The method of claim 19, wherein the microorganism host cell is selected from the species of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

21: The method of claim 15, wherein the microorganism host cell comprises an inactivated non-homologous end joining (NHEJ) system.

22: The method of claim 15, wherein the microorganism host cell comprises an inactivated DNA Ligase D (LigD) and/or DNA-end-binding protein Ku or homologoue(s) thereof.

23: The method of claim 15, wherein the microorganism host cell comprises an inactivated ligD, ku70 and/or ku80 gene or homologue(s) thereof.

24: The method of claim 15, wherein the polynucleotide-guided endonuclease is a Streptococcus pyogenes Cas9 or a homologue thereof.

25: The method of claim 15, wherein the polynucleotide-guided endonuclease has only one active nuclease domain.

26: The method of claim 15, wherein the polynucleotide-guided endonuclease is a Streptococcus pyogenes Cas9 variant comprising a substitution of aspartic acid for alanine in the amino acid position corresponding to position 10, D10A.

27: The method of claim 15, wherein the single-stranded oligonucleotide comprises at least 15 unmodified nucleotides on the opposite side of the cut or nick in the genome relative to the modification(s) and at least 15 unmodified nucleotides on the opposite side of the modification(s) relative to the cut or nick in the genome.

28: The method of claim 15, wherein the single-stranded oligonucleotide comprises at least 25 unmodified nucleotides on the opposite side of the cut or nick in the genome relative to the modification(s) and at least 25 unmodified nucleotides on the opposite side of the modification(s) relative to the cut or nick in the genome.

29: The method of claim 15, wherein the one or more desired nucleotide modification(s) comprises at least one insertion, deletion and/or substitution of one or more nucleotide or codon.

30: The method of any of claim 15, wherein at least two genome target sequences in the host cell are modified by at least one insertion, deletion and/or substitution of one or more nucleotide or codon.

31: The method of claim 15, wherein the at least one single-stranded oligonucleotide in addition to the one or more desired nucleotide modification(s) also comprises one or more mutation in the PAM or protospacer sequence, wherein said one or more mutation effectively blocks the polynucleotide-guided endonuclease when introduced into the target sequence.

32: The method of claim 15, wherein the microorganism host cell is transformed with a polynucleotide encoding a polypeptide of interest either before or after the steps in claim 15.

33: The method of claim 32, wherein the polypeptide of interest is an enzyme.

34: The method of claim 33, wherein the polypeptide of interest is a hydrolase, isomerase, ligase, lyase, oxidoreductase, or transferase.
